(19) United States
(12) Patent Application Publication
(10) Pub. No.: US 2019/0133126 A1
(43) Pub. Date: May 9, 2019

(54) Coatings and methods for infection-resistant medical devices

(71) Applicants: The Trustees of Columbia University in the City of New York, New York, NY (US); Bharati Vidyapeeth Deemed University, Pune, Maharashtra (IN)

(72) Inventors: Shanta M. Modak, Riveredge, NJ (US); Arnab Kumar Ghosh, North 24 Parganas (IN); Chathuranga C. De Silva, New York, NY (US); Mahabaleshwar Hegde, Pune, Maharashtra (IN); Anand Arvind Zanwar, Jalna, Maharashtra (IN); Santoshkumar Hanmantrao Dongre, Aurangabad, Maharashtra (IN); Shivajirao S. Kadam, Pune, Maharashtra (IN)

(21) Appl. No.: 16/099,799

(22) PCT Filed: May 10, 2017
(86) PCT No.: PCT/US17/31969

§ 371 Date: Nov. 8, 2018

Related U.S. Application Data
(60) Provisional application No. 62/333,905, filed on May 10, 2016.

(51) Int. Cl. A01N 43/54; A61L 29/16; A61K 9/06; A61K 9/00; A01N 33/00; A01N 35/02; A61K 33/24; A61K 31/121; A61K 31/155; A61K 47/22; A61K 47/10; A61K 47/12; A61K 47/34; A61L 29/04; A61L 29/06; A61P 31/04

(52) U.S. Cl. CPC A01N 43/54; A61L 2300/224; A61K 9/06; A61K 9/0014; A01N 33/00; A01N 35/02; A61K 33/24; A61K 31/121; A61K 31/155; A61K 47/22; A61K 47/10; A61K 47/12; A61K 47/34; A61L 29/04; A61L 29/06; A61P 31/04; A61L 2420/02; A61L 2300/216; A61L 2300/206; A61L 29/16

(57) ABSTRACT

Disclosed herein are compositions useful for coating medical devices which have antimicrobial (anti-infective) qualities and which are simple to manufacture and cost-effective, and therefore suitable for global use, including in developing countries with economic constraints, and in a cost-conscious healthcare environment. In one embodiment, disclosed are formulations that include chlorhexidine (CHX), curcumin (CUR), for example, curcumin C3 complex, and a silver (Ag) salt. Other embodiments pertain to compositions that include CHX, Ag and a lubricating agent. The antimicrobial coatings made according to embodiments of the invention are easier to produce, have superior efficacy and devices coated with this composition show initial release of antimicrobials and prolonged prevention of bacterial adherence, compared to currently available alternatives, to significantly reduce device-related infection especially catheter associated urinary tract infection.

### CROSS-REFERENCE TO RELATED APPLICATIONS

This application claims benefit of Provisional application. 62/333,905, filed May 10, 2016 the entire contents of which are hereby incorporated by reference as if fully set forth herein, under 35 U.S.C. § 119(e).

### BACKGROUND OF THE INVENTION

Medical devices for implant or insertion into the body of a patient are used commonly in medical practice. For example, implants are common in the fields of orthopedics, dentistry, plastic surgery, and many general uses such as various forms of catheters and indwelling tubing. Such catheters and tubing include urinary catheters, central venous catheters, endotracheal tubes, cannulae as well as wound dressings the like. These devices are very useful in the treatment of patients; however, incidence of infection related to, associated with, or caused by these implants or the procedures used for their placement in the body can render these devices potentially dangerous.

Common complications associated with medical devices for implant include infection and inflammation, often beginning with colonization of the implant surface or its surrounding area with bacteria or formation of a biofilm on the surface of the implant. Bacteria can cause chronic inflammation of the site around the implant, destruction of tissue, and even very serious acute problems like septicemia.

While some catheters or other medical devices have been coated with antimicrobial coatings to reduce bacterial growth, they are complex to manufacture and have demonstrated limited long-term clinical benefit. Furthermore, due to high manufacturing costs, many developing countries lack access to existing antimicrobial catheters or coated medical devices for implant. In summary, therefore, prior art medical devices with antimicrobial coatings have been significantly ineffective and largely unaffordable to the general public, rendering them unsuitable for the global market. Therefore, there is great demand for an affordable, safe, and effective method for antimicrobial coating on medical devices for implant, such as urinary catheters and indwelling tubing, to reduce potential infections.

### SUMMARY OF THE INVENTION

Therefore, there is a need in the art for improved methods and compositions to produce infection-resistant materials and objects for use in the medical field. According to certain embodiments, disclosed herein are antibacterial compositions that can be used to coat medical device articles to form a stable, effective and lubricious antimicrobial coating. Certain composition embodiments include a combination of chlorhexidine base (CHX), curcumin C3 complex (CUR), silver salt (Ag) i.e., CHX—CUR—Ag and at least one solvent. The CUR and CHX may be dissolved at a ratio between 1:1 and 1:2 in at least one solvent (typically at a pH of 6.5-7.0), and the silver salt is suspended or dissolved in the solvent depending on the solvent used and the pH. In a specific embodiment, a preferred solvent for use is tetrahydrofuran and/or alkanol.

The CHX—CUR—Ag containing composition may further include a biomedical polymer. The biomedical polymer may be selected from one or more of polyurethane (e.g. PU 93A such as EG-93A from Tecoflex™, polyurethane EG-60D Tecoflex™), silicone adhesive Type A or silicone adhesive MD7-4502 and combinations thereof. In a specific embodiment, the CHX—CUR—Ag-containing composition includes 60-85% v/v tetrahydrofuran, 0-30% v/v alkanol, 0.5-3.0% w/v chlorhexidine base, 0.25-2.0% w/v curcumin, 0.2-1.0% w/v silver salt, and 1.0-5.0% w/v biomedical polymer. It is noted that when the silver salt is silver nitrate, ammonium hydroxide is added to the composition, which serves to dissolve the silver nitrate.

The CHX—CUR—Ag-containing composition also can further include at least one lubricity enhancing agent. Examples of such lubricity enhancing agents include but are not limited to a natural oil such as flax seed oil, grape seed oil, cranberry oil, avocado oil and any combination thereof; an emollient solvent such as octoxyglycerin, and/or alkanediols; or a hydrogel such as polyethylene oxide and polyethylene glycol; or any combination thereof. In a specific embodiment, the lubricity enhancement agent is a natural oil (e.g. flax seed oil).

In other embodiments, methods for making antimicrobial compositions are provided. In a specific example, an antimicrobial composition is produced as follows:

(a) mixing chlorhexidine base and curcumin;

(b) adding biomedical polymer dissolved in tetrahydrofuran to the mixture of step (a);

(c) adding mandelic acid or lactic acid to the mixture of step (b) to adjust the pH to 6.5-7.0;

(d) in a separate container, mixing a silver salt and tetrahydrofuran to form a silver mixture; and
(e) adding the silver mixture of step (d) to the mixture of step (c).

In a more specific embodiment, the method may further include:

(f) optionally adding a lubricity-enhancing agent to the mixture of step (a), wherein the—enhancing agent comprises one or more oils selected from the group consisting of silicone oil, flax seed oil, grape seed oil, cranberry oil, and avocado oil; one or more emollient solvents selected from the group consisting of octoxyglycerin and alkanediols; or one or more hydrogels selected from the group consisting of polyethylene oxide and polyethylene glycol; wherein when the lubricating agent is an alkanediol, it is optionally dissolved in an aliphatic alcohol.

According to a more specific embodiment, provided is a method of making an antimicrobial composition that is produced as follows:

a) mixing chlorhexidine base and alkanol:
b) adding biomedical polymer dissolved in tetrahydrofuran to the mixture of step (a);

c) in a separate container, mixing silver nitrate and tetrahydrofuran to form silver mixture; and adding ammonium hydroxide to a final concentration of 0.5% to form a clear solution; and
d) adding the silver mixture of step (c) to the mixture of step (b).

And in a more specific embodiment, the method further involves:

(e) adding a lubricating agent to the mixture of step (a); wherein the lubricity-enhancing agent comprises one or more oils selected from the group consisting of silicone oil, flax seed oil, grape seed oil, cranberry oil, and avocado oil; one or more emollient solvents selected from the group consisting of octoxyglycerin and alkanediols; or one or more hydrogels selected from the group consisting of polyethylene oxide and polyethylene glycol; wherein when the lubricating agent is an alkanediol, it is optionally dissolved in an aliphatic alcohol.

In an even more specific embodiment, the lubricating agent is flax seed oil and the alkanol is methanol.

In another embodiment, a composition including CHX, a silver salt and at least one lubricity enhancing agent is provided. In a specific example, the lubricity enhancing agent is a natural oil. In an even more specific embodiment, the natural oil is flax seed oil. In a specific embodiment, the CHX—Ag plus lubricity enhancing agent composition includes 60-90% v/v tetrahydrofuran, 10-30% v/v alkanol, 0.5-3.0% w/v chlorhexidine base, 0.2-1.0% w/v silver salt, 1-7% v/v natural oil and 1-5% v/v biomedical polymer.

Embodiments also involve making a composition containing CHX, silver salt and a lubricity enhancing agent. In a specific embodiment, such composition is produced as follows: (a) mixing chlorhexidine base in methanol in a container; (b) adding biomedical polymer dissolved in tetrahydrofuran to the mixture of step (a); (c) adding a lubricity enhancing agent (e.g. flax seed oil) to the mixture of step (b); (d) in a separate container, mixing a silver salt and tetrahydrofuran to form silver mixture; and (e) adding the silver mixture of step (d) to the mixture of step (c).

According to another embodiment, a method for making a composition particularly suitable for coating a silicone catheter is provided. An example of such method involves the following steps:

(a) mixing chlorhexidine base and curcumin C3 complex in a container;

(b) adding a biomedical polymer mixture (for example, EG 93A and/or EG 60D) dissolved in tetrahydrofuran to the mixture of step (a);

(c) adding mandelic acid or lactic acid to the mixture of step (b) to adjust the pH to 6.0-7.0;

(d) adding silicone medical adhesive (Type A or MD7-4502) to the mixture of step (c)

(e) in a separate container, mixing a silver salt and tetrahydrofuran to form silver mixture;

and

(f) adding the silver mixture of step (e) to the mixture of step (d); and

(g) optionally adding a lubricating agent to the mixture of step (a), wherein the lubricity-enhancing agent comprises one or more oils selected from the group consisting of silicone oil, flax seed oil, grape seed oil, cranberry oil, and avocado oil; one or more emollient solvents selected from the group consisting of octoxyglycerin and alkanediols; or one or more hydrogels selected from the group consisting of polyethylene oxide and polyethylene glycol; wherein when the lubricating agent is an alkanediol it is optionally dissolved in an aliphatic alcohol.

Other embodiments pertain to medical device articles coated with antimicrobial compositions disclosed herein. In specific embodiments, the medical device article is a catheter (e.g. urinary catheter).

These and other features, aspects, and advantages of the present invention will become better understood with regard to the following description, examples and appended claims.

Specific embodiments of the invention include a composition for providing an antibacterial coating, which comprises: (a) a solvent; (b) a chlorhexidine compound selected from chlorhexidine base and chlorhexidine salt; (c) a silver compound selected from elemental silver and silver salt; (d) a curcumin compound, a lubricity enhancing agent, or both a curcumin compound and a lubricity enhancing agent; (e) a pH adjusting compound selected from an acid; and (f) a biomedical polymer.

Preferably, the solvent is tetrahydrofuran (THF) or THF and an alkanol, wherein the alkanol preferably is methanol, ethanol, propanol, isopropanol, 1,3-propanediol, 2-methyl-2 propanol, hexanol, or any combination thereof. In certain preferred embodiments, the solvent is THF or is a combination of THF and methanol.

Preferably, the chlorhexidine compound is chlorhexidine base. In addition, preferably, the silver compound is a silver salt, most preferably silver sulfadiazine or silver nitrate.

Preferably, the curcumin compound is curcumin or curcumin-C3 complex.

Preferable lubricity enhancing agents are selected from a natural oil, an emollient solvent, and a hydrogel. The natural oil preferably is selected from flax seed oil, grape seed oil, cranberry oil, avocado oil and any combination thereof. The emollient solvent preferably is selected from octoxyglycerin, an alkanediols, and any combination thereof. The alkanediol preferably is 1,2-decanediol. The hydrogel is selected from polyethylene oxide and polyethylene glycol, or any combination thereof.

In preferred compositions, the acid is selected from mandelic acid and lactic acid and the base is ammonium hydroxide.

Preferably, the biomedical polymer is selected from polyurethane EG-93A, polyurethane EG-60D, silicone adhesive Type A, silicone adhesive MD7-4502 and any combination thereof.

Generally preferred compositions contain 60-85% v/v tetrahydrofuran, 10-30% alkanol, 0.5-3.0% chlorhexidine base, 0.2-1.0% silver salt, 1-7% natural oil and 1-5% biomedical polymer, or contains 60-90% v/v tetrahydrofuran, 0-30% v/v alkanol, 0.5-3.0% w/v chlorhexidine base, 0.25-2.0% w/v curcumin, 0.2-1.0% w/v silver salt, and 1-5% w/v biomedical polymer.

Highly preferred compositions contain a chlorhexidine compound, a curcumin compound, silver sulfadiazine, 1,2-decanediol, and mandelic acid. Most preferred compositions are those in which the ratio of chlorhexidine compound to curcumin compound is 1:1 to 1:2.

The invention also encompasses embodiment in the form of an ointment, lotion, or cream comprising the composition of claim 1, and articles coated with the compositions described herein, including medical devices such as catheters, for example urinary catheters. Such articles preferably are made of latex or silicone.

The invention also encompasses embodiments including methods of making an article coated with compositions as described herein, which comprises soaking the article in a composition of claim 1. The soaking of the article in the compositions of the invention generally is for 30 seconds to 2 minutes.

For latex catheter where lubricating agent is present: Additional methods according to embodiments of the invention include methods of making the inventive compositions, which comprise the steps of:

(a) mixing together chlorhexidine base, mandelic acid lubricating agent 1,2 decanediol;

(b) adding methanol to the mixture of (a) to dissolve the ingredients

(c) adding a biomedical polymer EG 93A, dissolved in THF to the mixture of (b);

(d) adding curcumin C3 complex to the mixture of (c)

(e) in a separate container, mixing a silver salt and THF to form a silver mixture;

(f) adding the silver mixture of (e) to the mixture of (d)

For silicone catheter where lubricating agent is present: Additional methods according to embodiments of the invention include methods of making the inventive compositions, which comprise the steps of:

(a) mixing together chlorhexidine base, mandelic acid lubricating agent 1,2 decanediol;

(b) adding methanol to the mixture of (a) to dissolve the ingredients

(c) adding a biomedical polymer mixture of EG 93A & EG 60D, dissolved in THF to the mixture of (b);

(d) adding curcumin C3 complex to the mixture of (c)

(e) adding silicone medical adhesive Type A or MD7-4502 to the mixture of (d)

(f) in a separate container, mixing a silver salt and THF to form a silver mixture;

(g) adding the silver mixture of (f) to the mixture of (e)

### DETAILED DESCRIPTION

Disclosed herein are inventive embodiments designed to produce medical devices and coatings for medical devices which have antimicrobial (anti-infective) qualities and which are simple to manufacture and cost-effective, and therefore suitable for global use, including in developing countries with economic constraints, and in a cost-conscious healthcare environment. The core antimicrobial components of the invention are chlorhexidine (CHX), curcumin (CUR), for example, curcumin C3 complex, and silver (Ag) salt. Other embodiments pertain to compositions that include CHX, Ag and a lubricating agent, as well as methods of making and using the same. The antimicrobial coatings made according to embodiments of the invention are easier to produce, have superior efficacy and devices coated with this composition show initial release of antimicrobials and prolonged prevention of bacterial adherence, compared to currently available alternatives, to significantly reduce device-related infection especially catheter associated urinary tract infection. The three-part CHX—CUR—Ag formula also produces a lubricious surface as well as an antimicrobial one, thereby providing distinct and unexpected advantages over prior art products and coatings.

### 1. DEFINITIONS

Unless otherwise defined, all technical and scientific terms used herein are intended to have the same meaning as commonly understood in the art to which this invention pertains and at the time of its filing. Although various methods and materials similar or equivalent to those described herein can be used in the practice or testing of the present invention, suitable methods and materials are described below. However, the skilled should understand that the methods and materials used and described are examples and may not be the only ones suitable for use in the invention. Moreover, it should also be understood that as measurements are subject to inherent variability, any temperature, weight, volume, time interval, pH, salinity, molarity or molality, range, concentration and any other measurements, quantities or numerical expressions given herein are intended to be approximate and not exact or critical figures unless expressly stated to the contrary. Hence, where appropriate to the invention and as understood by those of skill in the art, it is proper to describe the various aspects of the invention using approximate or relative terms and terms of degree commonly employed in patent applications, such as: so dimensioned, about, approximately, substantially, essentially, consisting essentially of, comprising, and effective amount. Unless defined otherwise, all technical and scientific terms used herein have the same meaning as commonly understood by one of ordinary skill in the art.

The term “about,” as used herein, means plus or minus 20 percent of the recited value, so that, for example, “about 0.125” means 0.125±0.025, and “about 1.0” means 1.0±0.2.

The term “alkanol,” or “alkyl alcohol” as used herein, means an alcohol selected from methanol, ethanol, propanol, isopropanol, 1,3-propanediol, 2-methyl-2 propanol, hexanol, or combinations thereof. Aromatic alcohols, for example, but not by way of limitation, phenoxyethanol, benzyl alcohol, 1-phenoxy-2-propanol, and/or phenethyl alcohol, may also optionally be used in combination with aliphatic alcohols.

The term “CHX,” as used herein, refers to chlorhexidine (N,N″″1,6-hexanediylbis[N′-(4-chlorophenyl)(imidodicarbonimidic diamide), a cationic bisbiguanide compound:

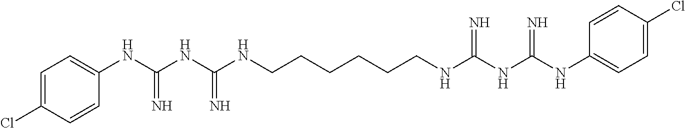 [SMILES: N=C(NCCCCCCNC(=N)NC(=N)Nc1ccc(Cl)cc1)NC(=N)Nc1ccc(Cl)cc1]

This term includes the base compound, shown above, and any salt thereof, for example chlorhexidine gluconate, chlorhexidine dihydrochloride, chlorhexidine diacetate and chlorhexidine digluconate. A non-inclusive list of further salts encompassed by this term is provided listed below. The base form is predominantly used with composition embodiments the present invention, however any available salt can be used as well.

The term “CUR” or “curcumin compound,” as used herein, refers, to curcumin ((1E, 6E)-1,7-Bis(4-hydroxy-3-methoxyphenyl)-1,6-heptadiene-3,5-dione), also known as diferuloylmethane, a tautomeric diarylheptanoid compound which exists in enolic form in organic solvents and as a keto compound in water:

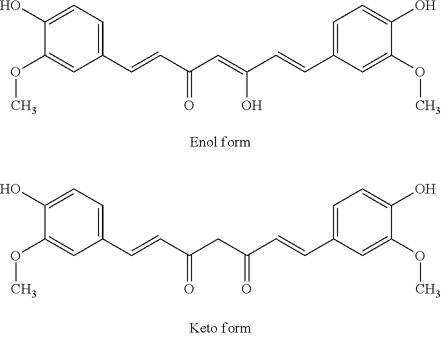 [SMILES: COc1cc(/C=C/C(=O)/C=C(O)/C=C/c2ccc(O)c(OC)c2)ccc1O ; COc1cc(/C=C/C(=O)CC(=O)/C=C/c2ccc(O)c(OC)c2)ccc1O]

This compound is found as the principle curcuminoid in turmeric, along with desmethoxycurcumin and bisdesmethoxycurcumin. Thus, as used herein, the terms “CUR” and “curcumin compound” also refer to these two additional compounds, and to mixtures of the three compounds. Mixtures of curcumin, desmethoxycurcumin, and bisdesmethoxycurcumin are sometimes referred to as C3 or cucumin-C3, and are included in the definition of “CUR” and “curcumin compound.” Other biologically compatible curcuminoids, including, but not limited to tetrahydrocurcumin, tetrahydrodesmethoxycurcumin, tetrahydrobisdemethoxycurcumin, and any combinations thereof, also are encompassed by the term “curcumin” or “CUR” herein unless specified otherwise. In one example, the curcumin used is curcumin-C3 complex (Sami Labs Ltd., Bangalore, India).

The term “silver” or “Ag” as used herein refers to elemental silver, or a silver salt. Any biologically compatible or pharmaceutically acceptable silver salt known in the art is appropriate and is included in the term “silver” or Ag.” Examples of suitable silver salts include, but are not limited to silver acetate, silver benzoate, silver bromate, silver carbonate, silver chlorate, silver chromate, silver citrate, silver fluoride, silver iodate, silver iodide, silver lactate, silver laurate, silver nitrate, silver oxide, silver palmitate, silver pentafluoropropionate, silver perchlorate, silver phosphate, silver protein, silver sulfadiazine, silver sulfate, silver p-toluene sulfonate, and the like. The silver or silver salt generally is provided at a concentration of about 0.1% to about 2% in the solution used for soaking or treating the articles to be produced, preferably about 0.1%, about 0.2%, about 0.5%, about 0.75%, about 1%, about 1.5% or about 2% in the solution, and most preferably about 0.2% to about 1.0% in the solution.

The terms “CHX—CUR—Ag” and “CHX—CUR—Ag composition,” as used herein, refer to a solution that includes chlorhexidine (CHX), curcumin (CUR), and silver or silver salt as defined herein (either as a suspension or a solution depending on the silver/salt and solvent/pH used). In a specific example, the CHX—CUR—Ag composition includes CUR and chlorhexidine base at a 1:1-1:2 ratio, dissolved in one or more organic solvents at pH 6.5-7.0 and a suspension of silver salt in this solution. The CHX—CUR—Ag composition also may contain optional lubricity-enhancing agents.

A “catheter,” as the term is used herein, means any basically tubular-shaped instrument, preferably flexible, which is designed to be passed through a body channel for withdrawing fluids from or inserting fluids into a body cavity. The term “catheter” includes the term cannula, which is a tube for insertion into a vessel, duct or cavity, usually for draining of fluids, or for administration of medical fluids such as oxygen gas, liquid medication, and the like. A “urinary catheter,” as the term is used herein, means a catheter for insertion into the urethra to collect urine from the urinary bladder.

The terms “coat” “coated” or “coating” (verb form) are used interchangeably herein and refer to the act of covering a surface with a composition for a sufficient period of time to impregnate the surface with the composition. When the composition is a solution of solute components in a solvent, coating may further involve drying the composition such that at least a portion of the solvent in the composition on the surface is removed and solute components remain on the surface.

The terms “coat” or “coating” (noun form) are used interchangeably herein to refer to a layer of material that covers a surface. In reference to a medical article, the entire surface or part of the surface may have a coating. The coat (noun) may include a CHX—CUR—Ag composition or CHX—Ag composition containing one or more lubricity enhancing agents on a surface or components of such compositions remaining on the surface after drying.

The term “coated” (in adjective form) as used herein refers to a surface having a coat.

The terms “coating solution,” or “coating composition” are used interchangeably herein, and refer to a solution or suspension containing a combination of CHX, CUR, and silver or CHX, silver and one or more lubricity enhancing agents in a form useful to coat a surface such as a medical device.

The term “solution” as used herein refers to a mixture composition of one or more solutes in a solvent. In addition to solute components, a solution as used herein, may include one or more constituents that are present as a suspension in the solution.

The term “infection-resistant,” as used herein, when referring to an item (e.g., a coated article), means that the item has the ability to reduce or retard adherence, growth or numbers of microorganisms per surface area than an uncoated (control) surface. Preferably, bacteria are reduced by at least 20%, at least 50%, at least 75% or at least 80%, and most preferably by at least 90%, at least 95%, at least 98% or at least 99% compared to control, in and/or on the “infection-resistant” item.

The term “lubricious,” as used herein in referring to a composition or a substance means that the composition or substance reduces the friction force of a surface treated, impregnated or coated therewith. A lubricious composition or substance typically possesses a smooth and slippery quality. The term “lubricity” as used herein is the property or state of being lubricious.

A “medical device article,” as the term is used herein, refers to any device inserted or designed to be inserted, partially or completely, into the body of a patient, or placed against an open wound on the body, temporarily (long or short term) or permanently, and includes, but is not limited to sutures, resorbable sutures, pins, rods, staples, screws, plates, clamps, dental implants, meshes, soft tissue patches, stents, drug implants, reservoirs or depots (such as drug depots, contraceptive implants (e.g., diaphragms, hormonal implants, vaginal rings, and the like), cardiovascular implants (e.g., pacemakers, heart valve replacements, arterial or venous stents, and the like), monitors (e.g., blood sugar monitors), artificial organs and parts thereof (e.g., peritoneal dialysis catheters, artificial pancreas, insulin pumps or implanted parts thereof, and the like), artificial joints, external ventricular drains and cerebral shunts, neural implants (e.g. cochlear implants, and the like), ostomy systems and parts thereof (e.g., colostomies, ileostomies, urostomies, and the like), dressings (e.g., wound dressings, dental and periodontal dressings, and the like), heart pacemakers, cannulae, needles, and catheters (e.g., urinary catheters, central venous lines, endotracheal tubes, tracheostomy tubes, naso- or oro-gastric tubes, and the like). Such implants can be made of metal (e.g., titanium, steel, platinum, platinum-iridium, and the like), polymers (e.g., silicone, latex, or the like), ceramic (e.g., apatite, silicon), carbon fiber, or any material suitable for implantation into the body.

A “patient,” as the term is used herein, means any subject in need of treatment by use of a medical device article, and includes but is not limited to any mammal, such as humans, companion animals, livestock, and the like.

### 2. OVERVIEW

Catheter-associated urinary tract infection (CAUTI) is the most common hospital acquired infection (HAI), accounting for 42% of all HAIs and leading to 8,000 deaths annually. Between 15 and 25% of hospitalized patients now receive urinary catheters (UC) and almost 85% of intensive care patients require UCs. UCs are also used frequently in older adults who are not hospitalized. The annual cost of hospitalization from indwelling catheterization is estimated to be S1.3 billion and the pressure on hospitals to reduce this cost is expected to rise, as the Centers for Medicare and Medicaid Services no longer allow hospitals to receive payments for CAUTI and other secondary infections. While the incidence of some types of nosocomial infections has dropped recently as the result of increased vigilance in response to this pressure, the incidence of CAUTI has quadrupled over the past decade and is expected to further increase with the projected rise in number of elderly patients requiring catheterization.

Studies of state-of-the-art antibacterial UCs coated with silver alloys and nitrofurazone have shown no clear clinical antimicrobial value and cost effectiveness. Incorporation of nitrofurazone impaired bacterial growth and adherence for only the first five days, while use of silver had only a minimal effect, these results coming at an increased cost of 80-130% over uncoated UCs. Even so, about 40% of hospitals currently employ antimicrobial UCs.

The benefits of infection-resistant medical devices of all types, including medical devices for implant, are clear. Since non-pharmacological intervention methods are preferred in order to reduce the risk of developing antibiotic-resistant organisms, there is obvious need and demand for new device coating technologies that are more effective in preventing CAUTI, and infections related to the wide range of medical devices for implant in use today.

In the case of urinary catheters, following catheterization, bacteria attach to the catheter surface extraluminally (between the surface of the urinary tract and the catheter surface) and intraluminally, the latter originating through the drainage system and adhering to the catheter lumen surface. Once attached, bacteria then rapidly multiply and colonize on the surface of the device, producing bacterial biofilm. Unless the colonized catheter is removed, the biofilm has the potential to re-seed the bladder with microorganism, causing a UTI. Therefore, some measure to avoid or reduce bacterial colonization of the catheter lumen is important to prevent migration of bacteria from the drainage system to the bladder.

An additional complication caused by bacterial colonization is blockage of lumen of the catheter due to encrustation. A rapid increase in pH caused by urease produced by the bacteria can cause crystals of magnesium and calcium phosphate to form in the urine. Complexes of these crystals and the bacteria adhere to the catheter surface and block the lumen.

Urinary catheters are the standard treatment for many common urological conditions. Implanted medical devices are becoming more and more common. Unfortunately, existing antimicrobial catheters are expensive to produce due to the second coating of lubricious material such as hydrogel needed on the surface to offer comfort to the patients during insertion. Furthermore, prolonged use of a catheter, such as a urinary catheter, or any medical device article, greatly increases the likelihood of inflammation or infection, such as catheter-associated urinary tract infection. These complications can be a major health concern in, for example, vulnerable populations such as the elderly, populations of developing countries, children and immunocompromised patients.

Embodiments of this invention relate to compositions for coating medical device articles to produce infection-resistant medical devices. Also provided are method embodiments for making these compositions and using these compositions to coat articles, and the articles and coatings. Certain embodiments disclosed herein provide low cost antimicrobial medical device articles, such as catheters, which provide a prolonged and broad spectrum antimicrobial efficacy, and methods for producing the same. This method is a simple one-step soaking method which coats antimicrobials on all surfaces of an article, including the outer surface as well as inner lumen of tubular articles simultaneously.

### 3. SUMMARY OF RESULTS

Multiple studies have been conducted using different formulations that include chlorhexidine base and a silver salt or a CHX—CUR—Ag composition. These studies evaluated antimicrobial efficacy and bacterial adherence of compositions applied to catheters or segments thereof. For example, latex urinary catheters coated in CHX, AgSD and PU93A containing compositions showed higher anti-microbial efficacy in zone of inhibition testing against P. aeruginosa compared to CHX and AgSD containing compositions without PU93A. In addition, the duration of retention of anti-microbial efficacy in latex urinary catheters coated in CHX, AgSD and PU93A containing compositions was the same for either 1% or 2% CHX in zone of inhibition testing. Latex urinary catheters coated with CHX, AgSD and PU93 containing compositions for 1 minute also showed same anti-microbial efficacy as catheters soaked for 5, 10, and 45 minutes. Furthermore, latex urinary catheters coated in compositions containing CHX in combination with different silver salts showed higher anti-microbial efficacy compared to CHX compositions without silver salt.

Latex urinary catheters coated with CHX (2%)+AgSD+EG-93A (2%) was more effective than CHX (2%)+EG-93A (2%) in preventing adherence of P. aeruginosa on catheter surface, latex urinary catheters coated with CHX (1%)+AgSD+EG-93A (2%) and CHX (2%)+AgSD+EG-93A (2%) showed almost equal antimicrobial efficacy in preventing adherence of common uro-pathogens on their surface, and latex urinary catheters coated with CHX and AgSD plus a lubricating agent selected from silicone adhesive Type A, silicone adhesive MD7-4502, octoxyglycerin and flax seed oil showed similar duration of anti-microbial efficacy over the course of 5 days with octoxyglycerin showing slightly higher anti-microbial activity on day 2. The flax seed oil group was more lubricious.

Toxicity studies on antimicrobial urinary catheters inserted into the external orifice of the vaginal opening in NZW rabbits with application of saline or cotton seed oil to vaginal mucosa showed no irritation or minimum irritation for catheters coated with CHX (1%)+AgSD+EG-93A (2%) and CHX (2%)+AgSD+EG-93A (2%) respectively, whereas uncoated catheters showed minimum irritation. The inner-luminal surface of latex urinary catheter coated with CHX (1%)+AgSD+EG-93A (2%) [Group 3] showed antimicrobial efficacy against Proteus mirabilis. Zone of inhibition tests showed that anti-microbial latex urinary catheter coated with CHX, silver sulfadiazine (AgSD) and PU93A was more effective than CHX, silver nitrate (AgNO₃) and PU93A against P. aeruginosa.

In additional tests, latex urinary catheters coated with CHX (1%)+AgNO₃+EG-93A (2%) [Group 8] were more effective than CHX (1%)+AgSD+EG-93A (2%) [Group 3] in preventing adherence of P. aeruginosa on catheter surface. Latex Foley catheters coated with CHX—CUR—Ag were more lubricious and show superior antimicrobial efficacy than that coated with CHX and Ag without curcumin, and latex Foley catheters coated with CHX-AgSD+5% FO were more lubricious and showed lowest bacterial growth of P. aeruginosa on the catheter surface than catheters coated with CHX-AgSD with 2% hydromer or 2% OG. Silicone catheters coated with CHX—CUR-AgSD were more lubricious and showed no bacterial growth P. aeruginosa or C. albicans on the catheter surface compared to CHX-AgSD coated catheters without CUR after 7 days.

In comparison testing, bacterial adherence on latex Foley catheters coated with CHX—CUR-AgSD versus commercial catheters using BactiGuard™ and uncoated catheters, the CHX—CUR-AgSD coating was found to be more effective in preventing adherence of E. coli, C. albicans, and P. aeruginosa.

Use of 1,2-decanediol as a lubricant on latex catheters improved adherence of the coating on the catheter surface, and did not negatively affect bacterial adherence prevention of the coating; use of octoxyglycerin lubricant also did not negatively affect the antimicrobial efficacy of the coating.

Semi-quantitative evaluation of bacterial colonies indicated that silicone urinary catheters coated with CHX+AgSD+CUR was found to be very effective in preventing the adherence of P. aeruginosa (clinical isolate) on its surface, even after 16 days, compared to uncoated catheters.

### 4. EMBODIMENTS OF THE INVENTION

Implantation or insertion of a medical device sometimes produces a rapid inflammatory reaction at the site, due to injury, immune reaction, bacterial colonization, or other factors. A biofilm may form, which can harbor microorganisms and encourage further adherence of bacteria on the device. Prevention of these phenomena or reduction of their effects is important in preventing medically significant infection. Embodiments of this invention are intended to assist in maintaining an environment around the implanted medical device that retards or reduces inflammation and infection by coating the medical device with a composition that imparts anti-infection properties.

In one embodiment, a method is disclosed of preparing a low cost antimicrobial catheter which provides a prolonged and broad spectrum antimicrobial efficacy. This method is a simple one-step soaking method which impregnates antimicrobials on the outer surface as well as inner lumen simultaneously. An example of a antimicrobial composition used for coating catheters contains a solvent such as 60-90% v/v THF and/or 10-30% v/v alkanol; 1.0-10.0% w/v biomedical polymer such as polyurethane polymer (EG-93A, EG-60D) and/or a silicone adhesive (e.g. silicone adhesive MD7-4502 or silicone adhesive Type A); and antimicrobial agents selected from 0.5-3.0% w/v chlorhexidine base (CHX), 0.25-2.0% w/v curcumin, 0.1-2.0% w/v silver salt (e.g., silver sulfadiazine, silver nitrate) and combinations thereof. Catheters or other medical devices coated with the compositions taught herein also represent useful embodiments.

A specific embodiment pertains to a CHX—CUR—Ag containing composition for rendering a medical device infection resistant and also making the surface lubricious. The device coated with the CHX—CUR—Ag composition can reduce inflammation caused by device implantation on the surrounding tissues since curcumin is known to have anti-inflammatory properties. This composition may further contain a lubricity enhancing agent. Examples of suitable lubricity enhancing agents include natural oils, such as flax seed oil, grape seed oil, cranberry oil, or avocado oil, silicone oils, emollient solvents (e.g., alkanediols, or octoxyglycerin), and hydrogels (e.g., polyethylene glycol). Implementation of natural oils in the coating compositions taught herein can also reduce the inflammation in the urinary tract due to catheterization. Catheters or another medical device coated with disclosed compositions can provide prolonged antibacterial activity as well as surface lubricity. In a specific embodiment, the lubricity enhancing agent is one or more of flax seed oil and 1,2 decanediol.

One advantage of catheters prepared according to methods described herein is that they are highly lubricious, making insertion easier and thus providing more comfort to the patient. Currently, catheters are made lubricous by applying a separate hydrogel coating. This extra step is time consuming and costly. One-step method embodiments described herein do not require an extra coating, and are therefore cheaper and easier to carry out.

In certain embodiments, coating solutions implemented to coat medical device articles include selected solvents. Solvents may be selected according to three criteria: 1) stability of the medical grade aliphatic polyether-based thermoplastic polyurethanes (EG-93A and EG-60D) in the solvent; 2) relative volatility at room temperature; and/or 3) relative solubility of the components in the formulation. Distortion of polyurethanes can occur in aliphatic esters and ketones including acetone, methyl ethyl ketone (MEK) and cyclohexanone. Polyurethanes dissolve completely in dimethyl formamide (DMF) and dimethyl sulfoxide (DMSO), however, they possess very low volatility at room temperature. Specific embodiments involve the use of the cyclic ether tetrahydrofuran (THF), which was determined to meet the noted criteria due to its high volatility and compatibility with polyurethane. Embodiments also involved using methanol to dissolve CHX after adjustment of pH at 6.5-7.0, such as to dissolve the optional lubricating agents. The use of these solvents allows for coating surfaces of a medical device, including both the outer and inner surfaces of hollow or tubular devices such as catheters, with the antimicrobial composition simultaneously. Optionally, non-toxic oils and/or emollients can be added to the coating to further increase lubricity.

Natural oils/or emollients enhance the lubricity of the surface, which in turn helps to slow down the microbial colonization on catheter and subsequently shows prolonged efficacy, thereby decreasing the frequency of catheter replacement, providing additional value. Furthermore, such coated devices do not require a separate application of hydrogel lubricant, thereby saving additional steps and cost. The inventive methods and products therefore are expected to be versatile and useful on any medical device article.

In addition to coating medical device articles, compositions taught herein may be used topically directly on skin or wounds to control infection. In such embodiments, the compositions are formulated to possess antimicrobial efficacy as wells as to minimize irritation to skin.

Alternative embodiments pertain to compositions that include CHX and silver in combination with a lubricity enhancing agent, without necessarily including CUR. Examples of suitable lubricity enhancing agents include natural oils, such as flax seed oil, grape seed oil, or avocado oil, silicone oils, emollient solvents (e.g., alkanediols, or octoxyglycerin), and hydrogels (e.g., polyethyleneglycol). Implementation of natural oils in the coating compositions taught herein can also reduce the inflammation in the urinary tract due to catheterization. It has been determined that including a natural oil, particularly flax seed oil, enhances the lubricity of the coated medical device article and increases the antimicrobial efficacy. A specific example of an antimicrobial composition used for impregnation of medical devices includes solvents such as 60-90% v/v THF and/or 10-30% v/v alkanol, 1.0-10.0% w/v; biomedical polymer such as polyurethane polymer (EG-93A or EG-60D) and/or silicone adhesive MD7-4502 or silicone adhesive Type A or combination of silicone adhesives; antimicrobial agents selected from 0.5-3.0% w/v chlorhexidine base (CHX), 0.1-2.0% w/v silver salt (e.g., silver sulfadiazine, silver nitrate); and 0.5-6.0% v/v natural oil. Catheters or other medical device articles coated with the above compositions also represent useful embodiments.

The invention advantageously can be used to produce a low-cost infection resistant urinary catheter for use in humans or in animals, including companion animals (e.g. dogs, cats, and the like), laboratory animals (e.g., mice, rats, rabbits, chimpanzees, and the like) and livestock (e.g., cattle, horses, sheep, and the like). Any article made up of either latex or silicone polymer that can be exposed to bacteria with the formation of a biofilm, or which can harbor bacteria can be coated according to the invention. In addition to medical device articles that are inserted into a patient, compositions of the present invention may also be applied to wound care items, such as, but not limited to, wound coverings, bandages, tape, and steri-strips, and medical articles such as medical gowns, caps, face masks, and shoe-covers, surgical drops, etc.

Infectious Agents: Disclosed embodiments can be useful for reduction of or prevention of infection by a wide range of gram-positive as well as gram negative bacteria, including, but not limited to gram positive Staphylococcus aureus, methicillin-resistant S. aureus (MRSA), Staphylococcus epidermidis, Enterococcus faecalis, Staphylococcus saprophyticus, vancomycin-resistant Enterococcus (VRE) spp. and Gram negative Escherichia coli, Klebsiella pneumoniae, Enterobacter spp., Pseudomonas aeruginosa, Proteus mirabilis, Citrobacter spp., and yeast, including but not limited to Candida albicans.

Chlorhexidine and chlorhexidine Salts: As noted above, reference to chlorhexidine, refers to chlorhexidine including its base form or as salt. Pharmaceutically acceptable chlorhexidine salts that may be used as antimicrobial agents according to the invention include, but are not limited to, chlorhexidine palmitate, chlorhexidine diphosphanilate, chlorhexidine digluconate, chlorhexidine diacetate, chlorhexidine dihydrochloride, chlorhexidine dichloride, chlorhexidine dihydroiodide, chlorhexidine diperchlorate, chlorhexidine dinitrate, chlorhexidine sulfate, chlorhexidine sulfite, chlorhexidine thiosulfate, chlorhexidine di-acid phosphate, chlorhexidine difluorophosphate, chlorhexidine diformate, chlorhexidine dipropionate, chlorhexidine di-iodobutyrate, chlorhexidine din-valerate, chlorhexidine dicaproate, chlorhexidine malonate, chlorhexidine succinate, chlorhexidine malate, chlorhexidine tartrate, chlorhexidine dimonoglycolate, chlorhexidine monodiglycolate, chlorhexidine dilactate, chlorhexidine di-α-hydroxyisobutyrate, chlorhexidine diglucoheptonate, chlorhexidine di-isothionate, chlorhexidine dibenzoate, chlorhexidine dicinnamate, chlorhexidine dimandelate, chlorhexidine di-isophthalate, chlorhexidine di-2-hydroxynapthoate, and chlorhexidine embonate. Chlorhexidine free base is a further example of an antimicrobial agent. These and further examples of antimicrobial agents useful in this invention can be found in such references as Goodman and Gilman's The Pharmacological Basis of Therapeutics (Goodman Gilman A, Rall T W, Nies A S, Taylor P, ed. (Pergamon Press; Elmsford, N.Y.: 1990), the contents of which are hereby incorporated by reference.

Compounds having other properties also can be added to the inventive solutions to improve various properties. For example, it is contemplated that in some embodiments compounds such as the following can optionally be added: preservatives, colorants, dyes, surfactants, antioxidants (e.g. vitamin E, vitamin C), solvents, fillers, pH adjusters, fragrances, and pharmaceuticals (e.g. povidone iodine quaternary ammonium compounds, nitrofurazone and anti-coagulants).

### Compositions and Methods Using CHX—CUR—Ag Composition

In general terms, embodiments of the invention provide a composition and method for producing medical device articles that are anti-infective. In certain embodiments, the articles are treated with a composition containing a three-part mixture of chlorhexidine, curcumin and silver in a solvent as described herein and referred to as the “CHX—CUR—Ag composition.” This composition is useful for coating articles such as medical devices for implant with a superior ability to prevent and retard bacterial colonization and growth, and also has lubricious properties.

The products also optionally can be made even more highly lubricious, when this quality is desirable, without the need for a separately applied hydrogel coating, using simple additives to the antimicrobial coating. Therefore, a one-step coating process results in a high quality antimicrobial lubricious product with additional lubricious coating material as well. However, in an alternative embodiment, a lubricious coating also can be added as a separate coating over the antimicrobial coating.

Certain embodiments of the invention include a three-part mixture of chlorhexidine, curcumin and silver, as well as solutions, suspensions, and coatings made therefrom, and articles having such coatings. Additional embodiments include methods of producing the three-part mixture, coating solutions containing the mixture, and methods of coating articles with these solutions.

In a specific embodiment, the invention involves a coating composition suitable for urinary catheters and other medical devices that have strong antimicrobial properties as well as lubricity to assist with the insertion of the catheter. In a specific embodiment, the coating composition comprises the CHX—CUR—Ag composition and optional lubricating agent in a solvent system comprising methanol and tetrahydrofuran, which is useful to impart both antimicrobial activity and lubricity to the articles which it coats, such as catheters. With addition of non-toxic oils aiding in lubricity and incorporation of antimicrobial agents, this technology produces a simple, one-step method of producing effective, safe, and comfortable urinary catheters at a low cost, and has the potential to significantly reduce bacterial infection in vulnerable patient populations and developing countries, filling an important niche in the medical device field.

It has been discovered that a composition of chlorhexidine, curcumin and silver has a superior ability to combat bacterial adhesion and growth on surfaces. See Example 23.

In addition to catheters, the CHX—CUR—Ag composition is suitable for coating multiple types of surfaces, such as wound dressings, metal and rubber tubing. Suitable solutions and suspensions useful to produce an antimicrobial and lubricious coating comprise the CHX—CUR—Ag composition in an organic solvent, preferably tetrahydrofuran or a mixture of tetrahydrofuran and methanol or reagent grade anhydrous ethanol. Specific composition embodiments comprise 60-85% v/v tetrahydrofuran and 10-30% v/v methanol or ethanol, and include 1-10% w/v; of a biomedical polymer such as polyurethane polymer (EG-93A, EG-60D) and/or silicone adhesive (silicone adhesive MD7-4502 and/or silicone adhesive Type A). The biomedical polymer helps to incorporate the antimicrobial components inside the lumen as well as on the outer surface of the catheter thereby allowing sustained release of antimicrobials for prolonged time period and subsequently enhancing the antimicrobial efficacy of the catheter. Additionally it also helps to provide surface lubricity. The antimicrobial composition typically comprises 0.5-3.0% w/v chlorhexidine base (CHX base), 0.25-2.0% w/v curcumin (e.g. curcumin-C3 complex), and 0.1-2.0% w/v silver salt (e.g. silver sulfadiazine or silver nitrate).

Optionally, the CHX—CUR—Ag compositions further contain lubricity-enhancing agents which can be selected from but are not limited to flax seed oil, grape seed oil, avocado oil or other natural oils, silicone oils, or emollient solvents (e.g., Procetyl™ 10 (PPG-10 Cetyl Ether), PPG-3 benzyl ether myristate, ethyl hexyl glycerine, and/or octoxyglycerin). Natural oils also can reduce the inflammation in the urinary tract due to catheterization. Thus, oils and emollients that have useful properties such as lubricity, anti-inflammatory activity, penetration-enhancing activity or other antimicrobial compounds are contemplated for use with certain embodiments of the invention. In a specific embodiment, the lubricity-enhancing agents are included in the solution containing CHX—CUR—Ag. In this manner, articles for which enhanced lubricity is advantageous, such as catheters, can be treated to impart antimicrobial activity and enhanced lubricity in one treatment or coating step, simplifying manufacture and reducing expense.

To aid in solubilizing the silver or silver salts, such as in the antimicrobial composition including CHX, silver nitrate and a lubricity enhancing agent (CHX—Ag composition), a strong or weak base may be added to the solvent system of the coating solution. One example of a suitable base useful for this purpose is ammonium hydroxide, which can be added at a concentration of about 0.2% to about 1.0%. For example, if silver nitrate is insoluble in the chosen organic solvent at the desired concentration, adding ammonium hydroxide solubilizes the silver nitrate agent to form a clear solution. A solution containing this composition does not require agitation during the soaking/coating process.

Preferred examples of lubricity-enhancing agents include emollient solvents and/or natural oils. Non-limiting examples of highly preferred optional compounds for increasing lubricity are emollient solvents such as octoxyglycerin, alkanediols, PPG-10 Cetyl Ether, PPG-3 benzyl ether myristate and the like, and natural or synthetic oils such as silicone oils, avocado oil, flax seed oil, and hydrogels such as polyethylene glycol, polyethylene oxide 0.5% to about 7.0%, or about 1.0% to about 6.0%, and in a more specific embodiment, from about 2.0 to about 5.0%. Oils, such as flax seed oil and the like can be added in amounts ranging from about 0.5% to about 7.0%, or about 1.0% to about 6.0%, and in a more specific embodiment, from about 2.0% to about 5.0%.

The silver or silver salt generally is provided at a concentration of about 0.1% to about 2% in the solution used for soaking or treating the articles to be produced, or about 0.1%, about 0.2%, about 0.5%, about 0.75%, about 1%, about 1.5% or about 2% in the solution, and in a more specific embodiment, about 0.2% to about 1.0% in the solution. Specific examples include silver sulfadiazine in an amount ranging from about 0.2% to about 0.75% w/v, silver nitrate in an amount ranging from about 0.2% to about 0.75% silver carbonate in an amount ranging from about 0.2% to about 0.75% or silver oxide in an amount ranging from about 0.2% to about 0.75% In a more specific example, the silver compound is silver sulfadiazine.

A specific example of a solvent system for use with the invention is a mixture of tetrahydrofuran and one or more alkanols, typically methanol, however, as discussed above, other suitable solvents for the components in the CHX—CUR—Ag and/or coating solutions can be used. For example, suitable solvents include, but are not limited to tetrahydrofuran, ethanol, methanol, propanol, isopropyl alcohol, benzyl alcohol, 1,3-propanediol, 2-methyl-2 propanol, hexanol, or combinations thereof. Aromatic alcohols, for example, but not by way of limitation, phenoxyethanol, benzyl alcohol, 1-phenoxy-2-propanol, and/or phenethyl alcohol, may also optionally be used in combination with aliphatic alcohols. In specific embodiments, the solvent involves use of an organic volatile solvent like tetrahydrofuran (THF) and aliphatic alcohol like methanol or ethanol. THF is a moderately polar solvent and can dissolve a wide range of non-polar and polar chemical compounds. THF is used to dissolve the polyurethane polymer (EG-93A, EG-60D) and methanol or ethanol is used to dissolve CHX after adjustment of pH at 6.5-7.0.

Emollient solvents also can improve the lubricity of the surface. Certain natural oils (e.g., flax seed oil, grape seed oil, avocado oil, or cranberry oil) also have desirable properties, such as lubricity and anti-inflammatory effects. Therefore, in some embodiments of the invention, such compounds are added to provide these functions.

In other embodiments, one or more biomedical polymers are included in the CHX—CUR—Ag composition. Suitable polymers include, but are not limited to polyurethane, (e.g. EG-93A or EG-60D), polylactic acid, polyglycolic acid, polycaprolactone, polyvinyl chloride, polyvinyl alcohol, silicone polymer, silicone adhesive (e.g. silicone adhesive Type A or silicone adhesive MD7-4502), urethane adhesive, and combinations thereof. In a specific embodiment, polyurethane EG 93A is used. It has been found that using a biomedical polymer dramatically increases the duration of the antimicrobial efficacy of the coating. In another specific embodiment related to coating silicone surfaces, such as silicone catheters, a combination of EG-93A or EG-60D (or both) mixed with silicone adhesive Type A or MD7-4502 (or both) is used in the coating solution that includes CHX—CUR—Ag. It is noted that in a more specific embodiment for coating silicone catheters, the polymer may be EG 93A or a combination of EG93A+EG60D.

In order to produce a coated medical device that is antimicrobial and lubricious, which is safe, more easily manufactured, and cost-effective, the devices are soaked in a solution containing the CHX—CUR—Ag composition, allowing all surfaces (including the inside surface of hollow or tubular items) to be coated with the active agents of the invention, and, optionally, also lubricity-enhancing agents. Agitation may be performed during the soaking. The articles to be treated can be exposed to or soaked in a single solution or suspension containing three-part CHX—CUR—Ag composition as described herein, and optionally containing lubricity-enhancing agents, or the articles can be soaked in sequence in two separate solutions or suspensions, the first containing the antimicrobial CHX—CUR—Ag composition and the second containing the lubricity-enhancing agents. In addition, if lubricity needs to be enhanced for the catheter surface, then lubricity enhancing agents optionally can be added in the same coating solution containing CHX—CUR—Ag composition. Alternatively, if enhanced lubricity is not a concern for a particular article, the lubricity-enhancing agents can be omitted from this process. Thus, in certain embodiments, this technology provides one-step coating method to produce antimicrobial coatings on medical devices that are both highly lubricious and efficient to produce, while also being safe and cost-effective.

When the coating solution includes constituents in suspension, the coating or soaking process preferably is performed with continuous agitation to keep any particles in suspension. If a clear solution is used, agitation is not required, but is optional.

### CHX+Silver+Natural Oil (NO)

It has been discovered that compositions containing CHX and silver in combination with a natural oil (CHX—Ag—NO compositions) possess high lubricity and anti-microbial efficacy even without including curcumin. Accordingly, certain embodiments pertain to CHX—Ag—NO compositions, methods of making such compositions, as well as methods of coating medical device articles and the coated articles using same. As noted above, the natural oils may include flax seed oil, grape seed oil, cranberry oil, or avocado oil. In a specific embodiment, the natural oil implemented is flax seed oil.

As described above for the CHX—CUR—Ag compositions, solvent systems may be used to mix the CHX—Ag components. Solvents for this purpose typically involve THF and/or one or more alkanols, but other suitable solvents for the components in the compositions can be used. Examples of suitable solvents which can be used to prepare the CHX—Ag—NO compositions include, but are not limited to, tetrahydrofuran, ethanol, methanol, propanol, isopropyl alcohol, benzyl alcohol, 1,3-propanediol, 2-methyl-2 propanol, hexanol, or combinations thereof. Aromatic alcohols, for example, but not by way of limitation, phenoxyethanol, benzyl alcohol, 1-phenoxy-2-propanol, and/or phenethyl alcohol, also optionally may be used in combination with aliphatic alcohols, and the like. In specific examples, the solvents include 60-85% v/v THF and 10-30% v/v alkanol or 70-95% v/v THF.

Similarly, a biomedical polymer may be used in the production of the CHX—Ag—NO compositions. In specific examples, polyurethane polymer (EG-93A or EG-60D) is used at a concentration of about 1-5% w/v.

The general scheme for preparing the CHX—Ag—NO compositions involves mixing an amount of CHX in a solvent such as an alkanol (e.g. methanol) in a container. To this mixture, an amount of a polymer is added (e.g. EG93A in tetrahydrofuran) and mixed well until a clear, uniform solution is obtained. An amount of natural oil is added to the CHX containing coating solution to form a CHX—NO composition. In a separate container, an amount of a silver salt is mixed in THF (or if silver nitrate is used, it is dissolved in ammonia hydroxide) to make a uniform mixture and combined with the CHX-NO mixture to form a CHX—Ag—NO composition.

Similar to that discussed above for the CHX—CUR—Ag complex containing compositions, medical device articles may be contacted (e.g. soaked) with solutions, mixtures or suspensions of a CHX—Ag—NO composition under conditions to allow impregnation of the CHX—Ag—NO on the surface to produce a coated medical device that is antimicrobial and lubricious, as well as safe, more easily manufactured, and cost-effective. In certain examples involving hollow or tubular medical device articles, contact is provided to allow the inside surface and outside surface to be coated with the active agents of the invention. The articles to be treated can be exposed to or soaked in a single solution or suspension containing CHX, Ag and natural oil, as described herein or the articles can also be soaked in sequence in two separate solutions or suspensions, the first containing the antimicrobial CHX and Ag components and the second containing the lubricity-enhancing agents. Typically, natural oil is added in the same coating solution containing CHX—Ag antimicrobial composition.

The one-step soaking method is a straightforward process that involves contacting the surface of a medical device with the CHX—Ag—NO composition for a sufficient period of time to impregnate the surface with the CHX—Ag—NO. Contact may be performed such as by soaking the medical device article. In a specific embodiment, provided is a method that involves coating urinary catheters to provide strong antimicrobial properties well as lubricity to assist with the insertion of the catheter. This one step-method also avoids the need for the time consuming and costly extra hydrogel coating step.

### Topical Application and Wound Treatment

The compositions of the present invention may be used to treat wound healing or surface infections. In various non-limiting embodiments, the present invention may be utilized in products for direct topical application such as topical creams and lotions, wound care products, burn wound cream, decubitus ulcer cream, and therapeutic ointments. In specific non-limiting embodiments, topically applied compositions contain chlorhexidine, silver salt (e.g., silver sulfadiazine), and optionally curcumin and/or natural oil. Optionally, benzyl alcohol, and 1,3 propanediol or octanediol or decanediol are included, which also enhance the antifungal activity.

In various non-limiting embodiments, compositions for topical application may comprise a number of agents used in creams, lotions or ointments. Non-limiting examples include a thickening and/or gelling agent such as stearyl alcohol, cationic hydroxy ethyl cellulose (Ucare™; JR30), hydroxy propyl methyl cellulose, hydroxy propyl cellulose (Klucel™), chitosan pyrrolidone carboxylate (Kytamer™), behenyl alcohol, zinc stearate, emulsifying waxes, including but not limited to Ineroquat™ and Polawax™, an addition polymer of acrylic acid, a resin such as Carbopol® ETD™ 2020, guar gum, acacia, acrylates/steareth-20 methacrylate copolymer, agar, algin, alginic acid, ammonium acrylate co-polymers, ammonium alginate, ammonium chloride, ammonium sulfate, amylopectin, attapulgite, bentonite, C9-15 alcohols, calcium acetate, calcium alginate, calcium carrageenan, calcium chloride, caprylic alcohol, carbomer 910, carbomer 934, carbomer 934P, carbomer 940, carbomer 941, carboxymethyl hydroxyethyl cellulose, carboxymethyl hydroxypropyl guar, carrageenan, cellulose, cellulose gum, cetearyl alcohol, cetyl alcohol, corn starch, damar, dextrin, dibenzlidine sorbitol, ethylene dihydrogenated tallowamide, ethylene diolamide, ethylene distearamide, gelatin, guar gum, guar hydroxypropyltrimonium chloride, hectorite, hyaluronic acid, hydrated silica, hydroxybutyl methylcellulose, hydroxyethylcellulose, hydroxyethyl ethylcellulose, hydroxyethyl stearamide-MIPA, isocetyl alcohol, isostearyl alcohol, karaya gum, kelp, lauryl alcohol, locust bean gum, magnesium aluminum silicate, magnesium silicate, magnesium trisilicate, methoxy PEG-22/dodecyl glycol copolymer, methylcellulose, microcrystalline cellulose, montmorillonite, myristyl alcohol, oat flour, oleyl alcohol, palm kernel alcohol, pectin, PEG-2M, PEG-5M, polyacrylic acid, polyvinyl alcohol, potassium alginate, potassium aluminum polyacrylate, potassium carrageenan, potassium chloride, potassium sulfate, potato starch, propylene glycol alginate, sodium acrylate/vinyl alcohol copolymer, sodium carboxymethyl dextran, sodium carrageenan, sodium cellulose sulfate, sodium chloride, sodium polymethacylate, sodium silicoaluminate, sodium sulfate, stearalkonium bentonite, stearalkonium hectorite, stearyl alcohol, tallow alcohol, TEA-hydrochloride, tragacanth gum, tridecyl alcohol, tromethamine magnesium aluminum silicate, wheat flour, wheat starch, xanthan gum, abietyl alcohol, acrylinoleic acid, aluminum behenate, aluminum caprylate, aluminum dilinoleate, aluminum salts, such as distearate, and aluminum isostearates, beeswax, behenamide, butadiene/acrylonitrile copolymer, C29-70 acid, calcium behenate, calcium stearate, candelilla wax, carnauba, ceresin, cholesterol, cholesterol hydroxystearate, coconut alcohol, copal, diglyceryl stearate malate, dihydroabietyl alcohol, dimethyl lauramine oleate, dodecanoic acid/cetearyl alcohol/glycol copolymer, erucamide, ethylcellulose, glyceryl triacetyl hydroxystearate, glyceryl tri-acetyl ricinolate, glycol dibehenate, glycol di-octanoate, glycol distearate, hexanediol distearate, hydrogenated C6-14 olefin polymers, hydrogenated castor oil, hydrogenated cottonseed oil, hydrogenated lard, hydrogenated menhaden oil, hydrogenated palm kernel glycerides, hydrogenated palm kernel oil, hydrogenated palm oil, hydrogenated polyisobutene, hydrogenated soybean oil, hydrogenated tallow amide, hydrogenated tallow glyceride, hydrogenated vegetable glyceride, hydrogenated vegetable oil, Japan wax, jojoba wax, lanolin alcohol, shea butter, lauramide, methyl dehydroabietate, methyl hydrogenated rosinate, methyl rosinate, methylstyrene/vinyltoluene copolymer, microcrystalline wax, montan acid wax, montan wax, myristyleicosanol, myristyloctadecanol, octadecene/maleic anhydrine copolymer, octyldodecyl stearoyl stearate, oleamide, oleostearine, ouricury wax, oxidized polyethylene, ozokerite, paraffin, pentaerythrityl hydrogenated rosinate, pentaerythrityl tetraoctanoate, pentaerythrityl rosinate, pentaerythrityl tetraabietate, pentaerythrityl tetrabehenate, pentaerythrityl tetraoleate, pentaerythrityl tetrastearate, ophthalmic anhydride/glycerin/glycidyl decanoate copolymer, ophthalmic/trimellitic/glycols copolymer, polybutene, polybutylene terephthalate, polydipentene, polyethylene, polyisobutene, polyisoprene, polyvinyl butyral, polyvinyl laurate, propylene glycol dicaprylate, propylene glycol dicocoate, propylene glycol diisononanoate, propylene glycol dilaurate, propylene glycol dipelargonate, propylene glycol distearate, propylene glycol diundecanoate, PVP/eiconsene copolymer, PVP/hexadecene copolymer, rice bran wax, stearalkonium bentonite, stearalkonium hectorite, stearamide, stearamide DEA-distearate, stearamide DIBA-stearate, stearamide MEA-stearate, stearone, stearyl erucamide, stearyl stearate, stearyl stearoyl stearate, synthetic beeswax, synthetic wax, trihydroxystearin, triisononanoin, triisostearin, tri-isostearyl trilinoleate, trilaurin, trilinoleic acid, trilinolein, trimyristin, triolein, tripalmitin, tristearin, zinc laurate, zinc myristate, zinc neodecanoate, zinc rosinate, and mixtures thereof. The gelling agents used in vehicles may be natural gelling agents such as natural gums, starches, pectins, agar and gelatin. Often, the gelling agents are based on polysaccharides or proteins; examples include but are not limited to guar gum, xanthan gum, alginic acid (E400), sodium alginate (E401), potassium alginate (E402), ammonium alginate (E403), calcium alginate (E404,-polysaccharides from brown algae), Agar (E406, a polysaccharide obtained from red seaweeds), carrageenan (E407, a polysaccharide obtained from red seaweeds), Locust bean gum (E410, a natural gum from the seeds of the carob tree), Pectin (E440, a polysaccharide obtained from apple or citrus-fruit), and gelatin (E441, made by partial hydrolysis of animal collagen).

Various embodiments may comprise a stabilizer. In a non-limiting example, sodium perborate is used as the stabilizing agent in an amount ranging from about 0.3 to about 1% w/w.

Various embodiments may further comprise a surfactant. The surfactant may be an anionic surfactant, a cationic surfactant, an ampholytic surfactant, or a nonionic surfactant. Examples of nonionic surfactants include polyethoxylates, fatty alcohols (e.g., ceteth-20 (a cetyl ether of polyethylene oxide having an average of about 20 ethylene oxide units) and other “BRIJ”® nonionic surfactants available from ICI Americas, Inc. (Wilmington, Del.), cocamidopropyl betaine, alkyl phenols, fatty acid esters of sorbitol, sorbitan, or polyoxyethylene sorbitan. Suitable anionic surfactants include ammonium lauryl sulfate and lauryl ether sulfosuccinate. Preferred surfactants include lauroyl ethylenediamine triacetic acid sodium salt, Pluronic™ F87, Masil™ SF-19 (BASF™) and incromide.

Water used in the formulations described herein is preferably deionized water having a neutral pH.

Various embodiments of the invention may comprise additional additives, including but not limited to a silicone fluid (such as dimethicone or cyclomethicone), a silicone emulsion, dyes, fragrances, pH adjusters, including basic pH adjusters such as ammonia, mono-, di- and tri-alkyl amines, mono-, di- and tri-alkanolamines, alkali metal and alkaline earth metal hydroxides (e.g., ammonia, sodium hydroxide, potassium hydroxide, lithium hydroxide, monoethanolamine, triethylamine, isopropylamine, diethanolamine and triethanolamine); acid pH adjusters such as mineral acids and polycarboxylic acids (e.g., hydrochloric acid, nitric acid, phosphoric acid, sulfuric acid, citric acid, glycolic acid, and lactic acid); vitamins such as vitamin A, vitamin E and vitamin C; polyamino acids and salts, such as ethylenediamine tetraacidic acid (EDTA), preservatives such as Germall™ plus and DMDM hydantoin, and sunscreens such as aminobenzoic acid, arobenzone, cinoxate, diioxybenzone, homosalate, menthyl anthranilate, octocrylene, octyl methoxycinnamate, octyl salicylate, oxybenzoate, padimate 0, phenylbenzimidazole, sulfonic acid, sulisobenzone, titanium dioxide, and trolamine salicylate.

Non-limiting examples of cream products may further contain white petrolatum (2-20%), fatty alcohol (2-20%), emollient (1-10%), emulsifying agent (0.5-10%), humectant (2-15%), preservative (0.1-0.5%), and deionized or distilled water q.s. 100%. Fatty alcohols include stearyl, alcohol, cetyl alcohol, lauryl alcohol, myristyl alcohol, and other known fatty alcohols. Emollients include isopropyl myristate, lanolin, lanolin derivatives, isopropyl palmitate, isopropyl stearate and other known emollients. Emulsifying agents include sodium mono-oleate and polyoxyl 40 stearate. Humectants include propylene glycol, sorbitol, or glycerine or mixture thereof. Suitable water soluble preservatives include parabens, sorbic acid, benzoic acid, diazolidinyl urea, and iodopropylbutylcarbamate (Germal™).

### 5. EXAMPLES

It is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims. Unless defined, otherwise, all technical and scientific terms used herein have the same meanings as commonly understood by one of ordinary skill in the art. Although any methods and materials similar or equivalent to those described herein can be used in the practice or testing of embodiments of the present invention, the preferred methods, devices, and materials are now described. All publications mentioned herein, are incorporated by reference in their entirety. Nothing herein is to be construed as an admission that the invention is not entitled to antedate such disclosure by virtue of prior invention.

### Example 1: Effect of Polymer Concentration in the Coating Solution on the Antimicrobial Efficacy of Urinary Catheters

The antimicrobial agents used in the following groups 1-4 are chlorhexidine base and silver sulfadiazine. Methods were as follows. One gram of CHX was taken in a glass container, and 10.75 ml methanol was added and mixed. To this mixture, 50 ml of 4% EG-93A polymer in tetrahydrofuran (THF) was added and mixed well until a clear, uniform solution was obtained (the percentages of EG-93A are adjusted by the percentage of the EG-93A/THF mixture that is mixed with the CHX/methanol mixture). In a separate container, 0.75 g of AgSD was taken and 10 ml of THF was added to make a uniform AgSD paste and then 10 ml THF was added to make a uniform suspension, which was transferred to the CHX-EG 93A polymer mixture using a pipette. The container was rinsed with 15.5 ml THF and transferred to the CHX-EG 93A polymer mixture solution and mixed well (to form a CHX—Ag coating solution). (V) indicates measured by volume.

| | |
|---|---|
| Ingredients (%) | Percentage |

Group 1

| | |
|---|---|
| Chlorhexidine base | 1.0 |
| Methanol | 10.75 (V) |
| Silver sulfadiazine | 0.75 |
| PU 93 A | — |
| Tetrahydrofuran (THF) | 87.5 (V) |

Group 2

| | |
|---|---|
| Chlorhexidine base | 1.0 |
| Methanol | 10.75 (V) |
| Silver sulfadiazine | 0.75 |
| EG-93A | 1.0 |
| Tetrahydrofuran (THF) | 86.5 (V) |

Group 3

| | |
|---|---|
| Chlorhexidine base | 1.0 |
| Methanol | 10.75 (V) |
| Silver sulfadiazine | 0.75 |
| EG-93A | 2.0 |
| Tetrahydrofuran (THF) | 85.5 (V) |

Group 4

| | |
|---|---|
| Chlorhexidine base | 1.0 |
| Methanol | 10.75 (V) |
| Silver sulfadiazine | 0.75 |
| EG-93A | 3.0 |
| Tetrahydrofuran (THF) | 84.5 (V) |

Latex urinary catheters were soaked in the above-mentioned coating solutions for 1 hour and tested as follows.

Test 1:

Zone of Inhibition Test: A 0.5 cm segment from each group of catheters was embedded vertically in trypticase soy agar (TSA) media. The surface of the agar plate was spread with 0.3 ml of 10⁷ colony forming units (CFU) per ml of Pseudomonas aeruginosa culture prior to implant the catheter into the agar plate. Then the plates were incubated at 37° C. for 24 hours. The diameters of zones of inhibition of bacterial growth around the catheter segments were measured.

TABLE 1
Latex Urinary Catheter: Zone of inhibition
test against P. aeruginosa.

Zone of inhibition against
P. aeruginosa* (in mm scale)

| | Day | Day | Day | Day | Day |
|---|---|---|---|---|---|
| Experimental groups | 1 | 2 | 3 | 4 | 5 |
| Group 1 | 12 | 0 | | | |
| Group 2 | 17 | 14 | 8 | 0 | |
| Group 3 | 18 | 14 | 13 | 12 | 10 |
| Group 4 | 18 | 14 | 14 | 12 | 10 |

*Catheter diameter (5 mm) is included in the above zone of inhibition test.

Conclusion:

Group 3 [CHX+AgSD+EG-93A (2%)] and Group 4 [CHX+AgSD+EG-93A (3%)] showed highest anti-microbial efficacy in zone of inhibition testing against P. aeruginosa.

### Example 2: Effect of Concentration of Chlorhexidine Base (CHX) on the Antimicrobial Efficacy of Catheters

Compositions were prepared as described for Example 1 with amounts of components altered to achieve the following composition examples:

| | |
|---|---|
| Ingredients (%) | Percentage |

Group 3

| | |
|---|---|
| Chlorhexidine base | 1.0 |
| Methanol | 10.75 (V) |
| Silver sulfadiazine | 0.75 |
| EG-93A | 2.0 |
| Tetrahydrofuran (THF) | 85.5 (V) |

Group 5

| | |
|---|---|
| Chlorhexidine base | 2.0 |
| Methanol | 10.75 (V) |
| Silver sulfadiazine | 0.75 |
| EG-93A | 2.0 |
| Tetrahydrofuran (THF) | 84.5 (V) |

Latex urinary catheters were soaked in the above-mentioned coating solutions for 1 hour.

Test 2:

Retention of antimicrobial efficacy of latex catheter by zone of inhibition test: The duration of retention of antimicrobial activity of latex catheter was expressed in days until the zone of inhibition exhibited ≥7 mm. The method for zone of inhibition test is the same as described in Test 1. The catheter diameter was 5 mm

TABLE 2
Latex Urinary Catheter: Duration of retention of antimicrobial
efficacy of latex catheter by zone of inhibition test.

| | Duration of retention of antimicrobial activity (days) | | | |
|---|---|---|---|---|
| Experimental groups | S. aureus | E. coli | P. aeruginosa | C. albicans |
| Group 3 | >10 | 8 | 5 | 6 |
| Group 5 | >10 | 8 | 6 | 7 |
| Bardex ™ I.C. | 1 | 0 | 0 | 0 |
| (C.R. Bard) | | | | |

*Catheter diameter (5 mm) is included in the above zone of inhibition test.

Conclusion:

Duration of retention of antimicrobial efficacy exhibited by Group 3 [CHX (1%)+AgSD+EG-93A (2%)] and Group 5 [CHX (2%)+AgSD+EG-93A (3%)] was almost the same as that evident from zone of inhibition test. On the other hand, the Bardex™ I.C. (C.R. Bard) catheter did not show anti-microbial activity in zone of inhibition test.

### Example 3: Effect of Duration of Soaking Time on the Antimicrobial Efficacy of Catheters

Latex catheters were soaked for different time durations shown below in the indicated anti-microbial coating solutions described above.

Test 3:

Retention of antimicrobial efficacy of latex catheter soaked for different time durations: Retention of antimicrobial activity of latex catheters, soaked for different time durations as indicated below was expressed in days until the zone of inhibition exhibited ≥7 mm. The method for zone of inhibition test is the same as described in Test 1.

TABLE 3
Zone of inhibition test: Retention of antimicrobial efficacy of latex catheter soaked
for different time durations.

| Soaking | Group 3 | | | | Group 5 | | | |
|---|---|---|---|---|---|---|---|---|
| Time | S. aureus | E. coli | P. aeruginosa | C. albicans | S. aureus | E. coli | P. aeruginosa | C. albicans |
| 1 Min | >10 | 8 | 5 | 6 | >10 | 8 | 6 | 7 |
| 5 Min | >10 | 8 | 5 | 5 | >10 | 9 | 6 | 7 |
| 10 Min | >10 | 8 | 5 | 7 | >10 | 8 | 6 | 8 |
| 45 Min | >10 | 8 | 5 | 7 | >10 | 8 | 6 | 8 |

*Catheter diameter (5 mm) is included in the above zone of inhibition test.

Conclusion:

Urinary catheters can be soaked for 1 min as evident from zone of inhibition test.

### Example 4: Effect of Silver Salt on the Antimicrobial Efficacy of Urinary Catheters

Compositions were prepared as described for Example 1 with amounts of components altered to achieve the following composition examples:

| | |
|---|---|
| Ingredients (%) | Percentage |

Group 5

| | |
|---|---|
| Chlorhexidine base | 2.0 |
| Methanol | 10.75 (V) |
| Silver sulfadiazine | 0.75 |
| EG-93A | 2.0 |
| Tetrahydrofuran (THF) | 84.5 (V) |

Group 6

| | |
|---|---|
| Chlorhexidine base | 2.0 |
| Methanol | 10.75 (V) |
| Silver carbonate | 0.3 |
| EG-93A | 2.0 |
| Tetrahydrofuran (THF) | 84.95 (V) |

Group 7

| | |
|---|---|
| Chlorhexidine base | 2.0 |
| Methanol | 10.70 (V) |
| Silver nitrate | 0.3 |
| Ammonium hydroxide* | 0.5 |
| EG-93A | 2.0 |
| Tetrahydrofuran (THF) | 84.5 (V) |

Group 8

| | |
|---|---|
| Chlorhexidine base | 2.0 |
| Methanol | 10.75 (V) |
| Silver salt | — |
| EG-93A | 2.0 |
| Tetrahydrofuran (THF) | 85.25 (V) |

(V) - Measured by volume;
*Ammonium hydroxide was added to the CHX- silver nitrate mixture.

Latex urinary catheters were soaked in the above-mentioned coating solutions for 1 min.

Test 4:

Effect of addition of silver salt on the antimicrobial efficacy of urinary catheters by zone of inhibition test: the method was the same as described in Test 1.

TABLE 4
Zone of inhibition test: Effect of addition of silver salt
on the antimicrobial efficacy of urinary catheters.

Zone of inhibition against
C. albicans* (in mm scale)

| | Day | Day | Day | Day | Day |
|---|---|---|---|---|---|
| Experimental groups | 1 | 2 | 3 | 4 | 5 |
| Group 5 | 18 | 14 | 13 | 12 | 9 |
| Group 6 | 18 | 14 | 12 | 10 | 8 |
| Group 7 | 18 | 12 | 12 | 10 | 9 |
| Group 8 | 18 | 12 | 9 | 7 | 0 |

*Catheter diameter (5 mm) is included in the above zone of inhibition test.

Conclusion:

the zone of inhibition test showed that addition of silver salts viz. silver sulfadiazine (AgSD), silver carbonate (Ag₂CO₃) or silver nitrate (AgNO₃) enhance the antimicrobial efficacy of the coated latex urinary catheter against Candida albicans.

### Example 5: Effect of Addition of Silver Sulfadiazine to Prevent Biofilm Formation on the Surface of Latex Urinary Catheter Exposed to Contaminated Urine

Latex urinary catheters were soaked in the indicated anti-microbial coating solutions for 1 minute (see Table 5).

Test 5:

Method of determination of bacterial adherence on catheter surface: antimicrobial coated latex urinary catheter segments (2 cm) sealed at both ends, were placed in a sterile culture tube individually and suspended in 2 ml of artificial urine contaminated with Pseudomonas aeruginosa (10⁴ cfu/ml). The tubes were kept at 37° C. in an incubator with agitation at low speed, overnight. Uncoated catheter segments were used as the control. Catheter segments then were transferred into fresh artificial urine contaminated with P. aeruginosa (10⁴ cfu/ml) every day. After 7 days, the catheter segments were removed from the tubes and blotted on tissue paper to drain out the residual artificial urine. They were rinsed twice in 10 ml sterile normal saline and blotted dry. Each catheter segment was then put in 4 ml DNF (drug neutralizing fluid) in a culture tube and sonicated for 20 minutes. A 0.5 ml aliquot from each tube was then plated on TSA plate and incubated for 24 hours. Results are presented below in Table 5.

TABLE 5
Bacterial (P. aeruginosa) adherence on the surface of latex
urinary catheters after 7 days soaking in artificial urine.

| | Log₁₀ growth |
|---|---|
| Experimental groups | per 2 cm catheter |
| Control | 10.99 |
| Group 5 | 1.06 |
| Group 7 | 10.73 |

Conclusion:

Latex urinary catheter coated with CHX (2%)+AgSD+EG-93A (2%) [Group 5] was more effective than CHX (2%)+EG-93A (2%) [Group 7] in preventing adherence of P. aeruginosa on catheter surface.

### Example 6: Effect of Concentration of Chlorhexidine Base (CHX) in Preventing Biofilm Formation on the Surface of Latex Urinary Catheter Exposed to Contaminated Urine

Test 6:

Effect of concentration of chlorhexidine base (CHX) to prevent the adherence of uro-pathogens on catheter surface: the method to determine the adherence of uro-pathogens on catheter surface is the same as described in Test 5, above.

TABLE 6A
Bacterial (P. aeruginosa) adherence on the surface of latex
urinary catheter after 7 days soaking in artificial urine.

| | Log₁₀ growth |
|---|---|
| Experimental groups | per 2 cm catheter |
| Control | 11.3 |
| Group 3 | 1.23 |
| Group 5 | 1.06 |
| Bardex ™ I.C. (C. R. Bard) | 10.4 |

TABLE 6B
Adherence of Proteus mirabilis on the surface of latex
urinary catheter after 19 days soaking in artificial urine.

| | Log₁₀ growth |
|---|---|
| Experimental groups | per 2 cm catheter |
| Control | 7.19 |
| Group 3 | 1.1 |
| Group 5 | 0.0 |
| Bardex ™ I.C. (C. R. Bard) | 6.77 |

TABLE 6C
Adherence of Vancomycin-resistant Enterococcus faecium
(VREF) on the surface of latex urinary catheter
after 18 days soaking in artificial urine.

| | Log₁₀ growth |
|---|---|
| Experimental groups | per 2 cm catheter |
| Control | 6.5 |
| Group 3 | 1.86 |
| Group 5 | 1.44 |
| Bardex ™ I.C. (C. R. Bard) | 5.94 |

TABLE 6D
Adherence of Candida albicans on the surface of latex urinary
catheter after 10 days soaking in artificial urine.

| | Log₁₀ growth |
|---|---|
| Experimental groups | per 2 cm catheter |
| Control | 6.34 |
| Group 3 | 0.3 |
| Group 5 | 0.3 |
| Bardex ™ I.C. (C. R. Bard) | 5.16 |

Conclusion:

Latex urinary catheter coated with CHX (1%)+AgSD+EG-93A (2%) [Group 3] and CHX (2%)+AgSD+EG-93A (3%) [Group 5] showed almost equal antimicrobial efficacy in preventing adherence of common uro-pathogens on their surface. On the other hand, Bardex™ I.C. (C.R. Bard) did not show significant anti-microbial activity in preventing adherence of common uro-pathogens on its surface.

### Example 7: Effect of Addition of Different Lubricating Agents Viz. Flax Seed Oil, Ethyl Hexyl Glycerine and Silicone Adhesive Type a and Silicone Adhesive MD7-4502 on the Antimicrobial Efficacy of Urinary Catheters

Latex urinary catheters were soaked in the anti-microbial coating solutions as indicated below in Table 7 for 1 minute. CHX was taken in a glass container, and 10.75 ml methanol was added and mixed. To this mixture, 50 ml of 4% EG-93A polymer in tetrahydrofuran (THF) was added, mixed well until a clear, uniform solution is obtained. The lubricating agents octoxyglycerin (2% v/v) or silicone medical adhesive Type A (2% v/v) or silicone adhesive MD7-4502 (2% v/v) or flax seed oil (2% v/v/or 5% v/v/) were added to the CHX-polymer mixture.

In a separate container, 0.75 g of AgSD was taken and 10 ml of THF was added to make a uniform AgSD paste and then 10 ml THF was added to make a uniform suspension, which was transferred to the solution containing mixture of CHX, EG 93A polymer and lubricating agent, using a pipette. The container was rinsed with 13.5 ml THF and transferred to the same mixture solution and mixed well (CHX—Ag complex coating solution containing lubricating agent).

Test 7:

Effect of addition of different lubricating agents on the antimicrobial efficacy of urinary catheters by zone of inhibition test: the method was the same as described in Test 1.

TABLE 7
Zone of inhibition test: Antimicrobial efficacy of latex
catheter containing different lubricating agents.

| | Zone of inhibition, | | | | |
|---|---|---|---|---|---|
| | P. aeruginosa* (mm scale) | | | | |
| | Day | Day | Day | Day | Day |
| Experimental groups | 1 | 2 | 3 | 4 | 5 |
| CHX (1.0%) + AgSD (0.75%) + | 17 | 15 | 13 | 12 | 10 |
| Octoxyglycerin (2.0%) + EG-93A | | | | | |
| (2%) | | | | | |
| CHX (1.0%) + AgSD (0.75%) + | 17 | 13 | 12 | 11 | 9 |
| silicone adhesive Type A (2.0%) + | | | | | |
| EG-93A (2%) | | | | | |
| CHX (1.0%) + AgSD (0.75%) + | 17 | 12 | 11 | 10 | 8 |
| silicone adhesive MD7-4502 | | | | | |
| (2.0%) + EG-93A (2%) | | | | | |
| CHX (1.0%) + AgSD (0.75%) + Flax | 17 | 13 | 11 | 10 | 9 |
| seed oil (2.0%) + EG-93A (2%) | | | | | |
| CHX (1.0%) + AgSD (0.75%) + Flax | 17 | 13 | 11 | 10 | 9 |
| seed oil (5.0%) + EG-93A (2%) | | | | | |

*Catheter diameter (5 mm) is included in the zone of inhibition.

Conclusion:

All the groups showed similar zone of inhibition on Day 1, but the group containing ethyl hexyl glycerine (octoxyglycerin) showed a slightly higher zone of inhibition on day 2 onwards against P. aeruginosa. Flax seed oil groups were more lubricious.

### Example 8: Identification of Nontoxic Concentration of Chlorhexidine in Soaking Solution by Toxicity Studies in Animals Using Urinary Catheters Coated with Solution Containing 1% and 2% CHX

Objective: The objective of the study was to establish the vaginal irritation potential of the polar extract as well as non-polar extract of the coated urinary catheters (i.e., the coating) when tested in NZW rabbits following the ISO: 10993-10:2010(E) Guidelines.

Preparation of Urinary Catheter Extract:

The test material (urinary catheters coated with a solution containing 1% or 2% CHX) was extracted in normal saline or cotton seed oil by incubating at 70° C. for 72 hours at a ratio of 2.0 gm catheter in 10 ml saline or oil. The extract was decanted into a dry sterile container and cooled. The extract was prepared shortly before dosing.

Mucous Membrane Irritation Test with Normal Saline (Polar) Extract and Cotton Seed Oil (Non Polar) Extract of the Test Material:

1.0 ml of the saline (polar) extract or the cotton seed oil (non-polar) extract of the urinary catheters was applied to the vaginal mucosa of each animal in respective group. The procedure was repeated at 24-hour intervals every day for minimum of 5 consecutive days.

Observations:

The application sites were observed for evidence of erythema, edema and necrosis (see Table 8). The external orifice of the vaginal opening and the perineum was examined each day prior to dosing for signs of discharge, erythema and edema. At the end of the study, the entire vagina was dissected, opened longitudinally and examined for signs of irritation, breaks in the epithelial layer of tissue and necrosis. Finally, histologically processed (embedded, sectioned and stained in hematoxylin and eosin) and evaluated microscopically by a pathologist. See Table 8 for results. Range of values: 0.0-0.9=No irritation; 1.0-4.0=Minimal irritation; 5.0-8.0=Mild irritation; 9.0-11.0=Moderate irritation; 12.0-16.0=Severe irritation.

TABLE 8
Effects of Coating on Irritation.

| | | | Micro | | |
|---|---|---|---|---|---|
| | Macro | | histopathologic | | |
| | observation | | observation | | |
| | | Non- | | Non- | |
| | Polar | polar | Polar | polar | Irritation |
| Extract Sample | solvent | solvent | solvent | solvent | score |
| urinary catheter | 0 | 0 | 2.58 | 2.42 | Minimal |
| uncoated (Control) | | | | | irritation |
| urinary catheter | 0 | 0 | 0.16 | 0.42 | No |
| 1% CHX + AgSD | | | | | irritation |
| urinary catheter | 0 | 0 | 1.75 | 1.50 | Minimal |
| 2% CHX + AgSD | | | | | irritation |

Conclusion:

The saline and cotton seed oil extracts of the urinary catheters coated with antimicrobial agent 1% and 2% caused no irritation or minimum irritation respectively, whereas uncoated catheter showed minimum irritation to the vaginal mucous membrane in New Zealand white rabbits, according to the ISO:10993-10:2010(E) Guidelines.

### Example 9: One Step Method for Outer and Luminal Surface Impregnation on Latex Urinary Catheter Using the Following Solution

Coating compositions were prepared as follows.

| | |
|---|---|
| Ingredients (%) | Percentage |

Group 3

| | |
|---|---|
| Chlorhexidine base | 1.0 |
| Methanol | 10.75 (V) |
| Silver sulfadiazine | 0.75 |
| EG-93A | 2.0 |
| Tetrahydrofuran (THF) | 85.5 (V) |

Group 5

| | |
|---|---|
| Chlorhexidine base | 2.0 |
| Methanol | 10.75 (V) |
| Silver sulfadiazine | 0.75 |
| EG-93A | 2.0 |
| Tetrahydrofuran (THF) | 84.5 (V) |

Latex urinary catheters were wiped with 70% ethanol to clean the surface and dried at room temperature for 5-10 minutes. The catheters then were soaked in the above-mentioned coating solutions for 1 minute and placed in fume hood overnight until completely dried.

### Example 10: Level of Chlorhexidine (CHX) and Silver (Ag) Content in Latex Urinary Catheter (1 Minute Soaking Time)

TABLE 9
Chlorhexidine level (initial as well as post 10 days soaking in normal
saline) and initial silver content in latex urinary catheter.

| | Initial CHX | Initial Ag | Level of CHX (μg CHX/cm |
|---|---|---|---|
| Catheter | Level (μg CHX/ | content (μg Ag/ | of catheter) after 10 days |
| groups | cm of catheter) | cm of catheter) | soaking in normal saline |
| Group 3 | 370 | 102 | 26.48 |
| Group 5 | 600 | — | 28.48 |

### Example 11: Antimicrobial Efficacy of Inner-Luminal Surface of Latex Urinary Catheter

Latex urinary catheters, soaked for 1 minute in the anti-microbial coating solution containing CHX (1%)+AgSD+EG-93A (2%) [Group 3] were evaluated for the antibacterial efficacy of the luminal surface.

Test 8:

Antimicrobial efficacy of inner-luminal surface of latex urinary catheter. The method was as follows. Antimicrobial coated latex urinary catheter was cut into small pieces (6 cm segments). About 100 ml of sterile artificial urine was passed dropwise throughout the inner lumen of the catheter. Then the outer surface of the catheter segments was wiped with pure ethyl alcohol (≥99.5%) to remove the outer coating, followed by wiping once with tetrahydrofuran (THF). Then inner lumen of the latex catheter was filled with 10⁵ cfu/ml of Proteus mirabilis culture using a syringe. The open end of the catheter segment was clipped and kept on a low speed incubator shaker at 37° C. overnight. Uncoated catheter segments were used as the control. After 24 hours, 0.1 ml of culture collected from the inner lumen was added to 0.5 ml of drug neutralizing fluid (DNF). After required dilution, a 0.5 ml aliquot was plated on a TSA plate and incubated overnight at 37° C. Growth was calculated and recorded in Table 10.

TABLE 10
Antimicrobial efficacy of inner-luminal
surface of latex urinary catheter.

| | Log₁₀ growth/ |
|---|---|
| Experimental groups | 0.1 ml of culture |
| Control | 5.47 |
| Group 3 | 0.0 |

Conclusion:

The inner-luminal surface of the latex urinary catheter coated with CHX (1%)+AgSD+EG-93A (2%) [Group 3] showed antimicrobial efficacy against Proteus mirabilis.

### Example 12: Development of an Impregnation Solution Containing Solubilized Silver Salt with CHX

Compositions were prepared according to Example 1, but silver nitrate was used as the silver salt and a clear solution was prepared using ammonium hydroxide.

Coating of Latex Urinary Catheter with CHX-AM AgNO₃ (ammonium silver nitrate) was performed as follows.

Group 9

| Ingredients (%) | Percentage |
|---|---|
| Chlorhexidine base | 1.0 |
| Methanol | 15.75 (V) |
| Silver nitrate | 0.3 |
| Ammonium hydroxide | 0.5 |
| EG-93A | 2.0 |
| Tetrahydrofuran (THF) | 80.45 (V) |

(V) - Measured by volume

Latex urinary catheters were cut into small pieces (5 cm length). They were wiped with 70% ethanol to clean the surface and dried at room temp for 5-10 minutes. Finally, the catheters were soaked in the coating solution for 1 minute. They were placed in fume hood in hanging condition for overnight until completely dried.

### Example 13: Effect of the Silver Salts Viz. Silver Sulfadiazine or Silver Nitrate on the Antimicrobial Efficacy of Urinary Catheters

Latex urinary catheters were soaked for 1 minute in the anti-microbial coating solutions containing CHX (1%)+AgSD+EG-93A (2%) [Group 3] and CHX (1%)+AgNO₃+EG-93A (2%) [Group 9] separately.

Test 9:

the effect of different silver salts on the antimicrobial efficacy of urinary catheters was examined by zone of inhibition test: the method for the zone of inhibition test was the same as described for Test 1.

TABLE 11
Zone of inhibition test: Antimicrobial efficacy of
latex catheter containing different silver salts.

| | Zone of inhibition against | | | |
|---|---|---|---|---|
| | P. aeruginosa* (in mm scale) | | | |
| | Day | Day | Day | Day |
| Experimental groups | 1 | 2 | 3 | 4 |
| Chlorhexidine (CHX, 1.0%) + | 18 | 14 | 12 | 11 |
| Silver sulfadiazine (AgSD, | | | | |
| 0.75%) + EG-93A (2%) [Group 3] | | | | |
| CHX (1.0%) + Silver nitrate | 15 | 13 | 9 | 0 |
| (AgNO₃, 0.3%) + | | | | |
| EG-93A (2%) [Group 9] | | | | |

*Catheter diameter (5 mm) is included in the zone of inhibition test.

Conclusion:

the zone of inhibition test showed that anti-microbial latex urinary catheters containing silver sulfadiazine (AgSD) were more effective than silver nitrate (AgNO₃) against P. aeruginosa.

### Example 14: Effect of the Silver Salts Viz. Silver Sulfadiazine or Silver Nitrate to Prevent Bacterial Adherence on the Surface of Latex Urinary Catheter Exposed to Contaminated Urine

Latex urinary catheters were soaked in the anti-microbial coating solutions for 1 minute.

Test 10:

the effect of silver salts to prevent biofilm formation on the surface of latex urinary catheter was determined for the adherence of P. aeruginosa on the catheter surface using the same method as described in Test 5.

TABLE 12
Adherence of P. aeruginosa on the surface of latex urinary
catheter after 7 days soaking in artificial urine.

| | Log₁₀ growth |
|---|---|
| Experimental groups | per 2 cm catheter |
| Control | 11.28 |
| CHX (1%) + AgSD + EG-93A (2%) [Group 3] | 1.31 |
| CHX (1%) + AgNO₃ + EG-93A (2%) [Group 9] | 0.93 |

Conclusion:

coating latex urinary catheters with CHX (1%)+AgNO₃+EG-93A (2%) [Group 9] was more effective than CHX (1%)+AgSD+EG-93A (2%) [Group 3] in preventing adherence of P. aeruginosa on catheter surface.

### Example 15: Method of Preparing Coating Solution Containing CHX—CUR (Chlorhexidine and Curcumin)

One gm of CHX was taken in a glass container, and 1.0 gm (1:1 ratio of CHX to CUR) or 0.5 g (2:1 ratio of CHX to CUR) of curcumin-C3 complex was added to it. To this mixture, 50 ml of 4% EG-93A polymer in tetrahydrofuran (THF) was added, and finally 0.75 gm of mandelic acid was added to adjust the pH of the solution to 6.0-7.0, which was mixed well to produce a bright yellow colored CHX—CUR containing composition.

### Example 16: Preparation of Coating Solution Containing CHX—CUR—Ag (Chlorhexidine, Curcumin and Silver Sulfadiazine) for Latex Foley Catheters

CHX—CUR—Ag coating solution was prepared as follows:

| | Percentage (%) for 100 ml |
|---|---|
| Ingredients | coating solution |
| CHX | 1.0 |
| Mandelic acid | 0.75 |
| Curcumin C3 complex | 0.5 |
| EG-93A | 2.0 |
| Silver sulfadiazine (AgSD) | 0.75 |
| THF | 95.0 (V) |

(V) = by volume

One gm of CHX was taken in a glass container, and 1.0 gm (1:1 ratio of CHX to CUR) or 0.5 g (2:1 ratio of CHX to CUR) of curcumin-C3 complex was added to it. To this mixture, 50 ml of 4% EG-93A polymer in tetrahydrofuran (THF) was added, and finally 0.75 gm of mandelic acid was added to adjust the pH of the solution to 6.0-7.0, which was mixed well to produce a bright yellow colored CHX—CUR containing composition.

Alternatively, if an optional lubricating agent like decanediol was considered for addition to the antimicrobial coating composition to enhance the lubricity of the urinary catheter surface, then 1,2 decanediol (1.0% or 2%) was first dissolved in aliphatic alcohol, preferably in methanol (10%). Then 1.0 gm of CHX and 1.0 gm (1:1 ratio of CHX to CUR) or 0.5 g (2:1 ratio of CHX to CUR) of curcumin-C3 complex was added to it. To this mixture, 50 ml of 4% EG-93A polymer in tetrahydrofuran (THF) was added, and finally 0.75 gm of mandelic acid was added to adjust the pH of the solution to 6.0-7.0, which was mixed well to produce a bright yellow colored CHX—CUR.

In a separate container 0.75 g of AgSD was taken and 10 ml of THF was added to make a uniform paste of AgSD and then 10 ml THF was added to make a uniform solution and transferred to the CHX—CUR polymer solution using a pipette. The container was rinsed with 25 ml THF and transferred to the polymer solution and mixed well (CHX—CUR—Ag coating solution).

Method of coating latex urinary Foley catheter: the above CHX—CUR—Ag coating solution was transferred to a glass beaker. The catheter was placed in the coating solution and soaked for 1 minute, with continuous swirling of the catheter in a swift to prevent the AgSD from settling down. After 1 minute, the catheters were removed at a constant steady speed from the coating solution and allow to dry for 18 hours at room temperature.

The following groups were prepared.

| | |
|---|---|
| Ingredients (%) | Percentage (%) |

Group-10

| | |
|---|---|
| Chlorhexidine base | 1.0 |
| Mandelic acid | 0.75 |
| Silver sulfadiazine | 0.75 |
| EG-93A | 2.0 |
| Tetrahydrofuran (THF) | 95.5 (V) |

Group-11

| | |
|---|---|
| Chlorhexidine base | 1.0 |
| Mandelic acid | 0.75 |
| Curcumin C3-complex | 0.5 |
| Silver sulfadiazine | 0.75 |
| EG-93A | 2.0 |
| Tetrahydrofuran (THF) | 95.0 (V) |

Group-12

| | |
|---|---|
| Chlorhexidine base | 1.0 |
| Mandelic acid | 0.75 |
| Curcumin C3-complex | 1.0 |
| Silver sulfadiazine | 0.75 |
| EG-93A | 2.0 |
| Tetrahydrofuran (THF) | 94.5 (V) |

(V) - Measured by volume

### Example 17: Evaluation of Bacterial Adherence on the Surface of Coated Catheters

Evaluation of bacterial colonies was performed in artificial urine culture and bacterial adherence was determined on the above catheters soaked in urine culture using Test 5 described above (7 days in urine), using test organism: P. aeruginosa (10⁴ cfu/ml). For comparison, a commercial urinary catheter coated with silver, palladium and gold alloy (Bactiguard™) was also tested. Results are shown in Table 13, below.

TABLE 13
Adherence of P. aeruginosa on the surface of latex urinary
catheter after 7 days soaking in artificial urine

| | Bacterial | Log growth |
|---|---|---|
| | colonies | on 2 cm |
| | (cfu/ml) in | catheter |
| Groups | urine culture | segment |
| Uncoated catheter (control) | >10⁸ | 11.0 |
| 10 | 10⁴ | 1.2 |
| 11 | 0 | 0 |
| 12 | 0 | 0 |
| Bactiguard ™ | >10⁸ | 9.0 |

Conclusion:

Catheters coated with CHX—CUR—Ag were more lubricious and showed superior efficacy than those in the without curcumin group. Commercial Bactiguard™ was not effective.

### Example 18: Effect of Various Lubricating Agents in the CHX-AgSD Coating Solution on the Lubricity of Catheters

The following catheter groups containing lubricating agents were prepared. The coating compositions were prepared according to Example 1 with lubricating agents being added separately to the coating solution and mixed. Lubricating agents used were octoxyglycerin (OG) or flax seed oil (FO) and PEO Hydrogel (Hydromer). Catheters were soaked for 1 minute in this solution, removed at a steady speed and dried.

Group 10: 1.0% CHX+0.75% AgSD+2% EG-93A

Group 13: 1.0% CHX+0.75% AgSD+2% EG-93A+2% FO

Group 14: 1.0% CHX+0.75% AgSD+2% EG-93A+5% FO

Group 15: 1.0% CHX+0.75% AgSD+2% EG-93A+2% Hydromer

Group 16: 1.0% CHX+0.75% AgSD+2% EG-93A+2% OG

Evaluation of bacterial colonies in artificial urine culture and bacterial adherence on the above catheters soaked in urine culture was performed using Test 5 described above (7 days in urine culture), using test organism P. aeruginosa. In order to evaluate the relative efficacy of various catheter groups, a higher concentration (10⁶ cfu/ml) of culture was used for challenge.

TABLE 14
CHX-AgSD + lubricating agents (bacterial adherence).

| | Bacterial | Log growth |
|---|---|---|
| | colonies | on 2 cm |
| | (cfu/ml) in | catheter |
| Groups | urine culture | segment |
| Uncoated catheter (Control) | >10⁸ | 11.0 |
| 10 | 10³-10⁴ | 3.4 |
| 13 | 10³-10⁴ | 2.0 |
| 14 | 10³-10⁴ | 1.0 |
| 15 | 10³-10⁴ | 2.0 |
| 16 | 10³-10⁴ | 2.5 |

Conclusion:

Catheters containing CHX-AgSD+5% FO were more lubricious and showed lowest bacterial growth on the catheter surface.

### Example 19: Effect of Various Lubricating Agents in the Coating Solution Containing CHX—CUR-AgSD on the Lubricity of Catheters

Coating compositions were prepared according to Example 1 with the lubricating agents being added separately to the coating solution and mixed. Catheters were soaked for 1 minute in this solution, removed at a steady speed and dried.

Group 11. 1.0% CHX+0.5% CUR+0.75% AgSD+2% EG-93A

Group 17. 1.0% CHX+0.5% CUR+0.75% AgSD+2% EG-93A+2% OG

Group 18. 1.0% CHX+0.5% CUR+0.75% AgSD+2% EG-93A+2% FO

Group 19. 1.0% CHX+0.5% CUR+0.75% AgSD+2% EG-93A+5% FO

Group 20. 1.0% CHX+0.5% CUR+0.75% AgSD+2% EG-93A+2% Hydromer

Evaluation of bacterial colonies in artificial urine culture and bacterial adherence on the above catheters soaked in urine culture was performed using Test 5 described above (7 days in urine culture) using test organism P. aeruginosa. In order to evaluate the relative efficacy of various catheter groups, higher concentration (10⁶ cfu/ml) of culture was used for challenge.

TABLE 15
CHX-CUR-AgSD + lubricating agents (bacterial adherence).

| | Bacterial | Log growth |
|---|---|---|
| | colonies | on 2 cm |
| | (cfu/ml) in | catheter |
| Group | urine culture | segment |
| Uncoated catheter (Control) | >10⁸ | 11 |
| 11 | 0 | 0.0 |
| 17 | 10⁴ | 3.0 |
| 18 | 0 | 0.0 |
| 19 | 0 | 0.0 |
| 20 | 10² | 1.0 |

Conclusion:

Catheters containing CHX—CUR-AgSD or CHX—CUR-AgSD+FO were more lubricious and showed no bacterial growth on the catheter surface after 7 days.

### Example 20: Preparation of Coating Solutions Containing CHX—CUR—Ag

(Chlorhexidine, Silver sulfadiazine and curcumin composition) for silicone Foley catheters.

CHX—CUR—Ag coating solutions, were prepared as follows.

| | Percentage (%) for 100 ml |
|---|---|
| Ingredients | coating solution |
| CHX | 1.0 |
| Curcumin C3 complex | 1.0 |
| Mandelic Acid/Lactic acid | 0.75 |
| Polymer mix (EG-93A + EG-60D) | 2.0 |
| Medical adhesive type A-100/adhesive 4502 | 3.0 |
| AgSD | 0.75 |
| THF | 91.5 (V) |

(V) indicates measured in volume

Method of Preparation of 100 ml CHX—CUR—Ag Coating Solution:

One gm of CHX was taken in a glass container, and 1.0 gm (1:1 ratio of CHX to CUR) or 0.5 g (2:1 ratio of CHX to CUR) of curcumin-C3 complex was added to it. To this mixture, 24 ml of 8.3% polymer mixture (EG 93A+EG 60D (4:1) in tetrahydrofuran) was added and mixed well by vortex. Then 0.75 gm of mandelic acid was added to adjust the pH of the solution to 6.0-7.0, which was mixed well to produce a bright yellow colored CHX—CUR mixture in a polymer solution. To this solution, 3.0 gm of silicone medical adhesive Type A-100 or Silicone adhesive MD7-4502 was added and mixed well. For coating silicone catheters, the polymer may be EG 93A or a combination of EG93A+EG60D.

Alternatively, if an optional lubricating agent like decanediol was considered for addition to the antimicrobial coating composition to enhance the lubricity of the urinary catheter surface, then decanediol (1.0% or 2%) was first dissolved in aliphatic alcohol, preferably in methanol (10%). Then 1.0 gm of CHX and 1.0 gm (1:1 ratio of CHX to CUR) or 0.5 g (2:1 ratio of CHX to CUR) of curcumin-C3 complex was added to it. To this mixture, 24 ml of 8.3% polymer mixture (EG 93A+EG 60D (4:1) in tetrahydrofuran) was added and mixed well by vortex. Then 0.75 gm of mandelic acid was added to adjust the pH of the solution to 6.0-7.0, and mixed well to produce a bright yellow colored CHX—CUR mixture in a polymer solution. To this solution, 3.0 gm of silicone medical adhesive Type A-100/Silicone adhesive MD7-4502 was added and mixed well.

In a separate container, 0.75 g of AgSD was taken and 10 ml of THF was added to make a uniform AgSD paste and then 10 ml THF was added to make a uniform solution which was transferred to the CHX—CUR polymer solution using a pipette. The container was rinsed with 45 ml THF and transferred to the polymer solution and mixed well to form the CHX—CUR—Ag coating solution.

The method for coating silicone catheters was the same as described above for latex catheters.

### Example 21: Silicone Catheters Coated with Following Groups were Prepared

The coating compositions were prepared according to Example 20 with the lubricating agents being added separately to the coating solution and mixed. Catheters were soaked for 1 minute in this solution, removed at a steady speed and dried.

### Group 21: 1.0% CHX+1.0% CUR+0.75% AgSD+2% Polymer mix (EG 93A+60D)

### Group 22: 1.0% CHX+1.0% CUR+0.75% AgSD+2% Polymer mix (EG 93A+60D)+FO

### Group 23: 1.0% CHX+1.0% CUR+0.75% AgSD+2% Polymer mix (EG 93A+60D)+1% Decanediol

### Group 24: 1.0% CHX+0.75% AgSD+2% Polymer mix (EG 93A+60D)

Evaluation of bacterial colonies was performed in artificial urine culture and bacterial adherence was determined on the above catheters soaked in urine culture using Test 5 described above (7 days in urine) using test organism P. aeruginosa (10⁵ cfu/ml).

TABLE 16
Effect of curcumin C-3 complex and lubricating
agents on CHX-AgSD coated silicone catheter.

| | Bacterial | Log growth |
|---|---|---|
| | colonies | on 2 cm |
| | (cfu/ml) in | catheter |
| Groups | urine culture | segment |
| Uncoated catheter (Control) | >10⁷ | 10.0 |
| 21 | 0 | 0 |
| 22 | 0 | 0 |
| 23 | 0 | 0 |
| 24 | 10²-10³ | 2.0 |

Conclusion:

Silicone catheters containing CHX—CUR-AgSD were more lubricious and showed no bacterial growth on the catheter surface after 7 days.

### Example 22: Evaluation of Microbial Colonies in Artificial Urine Culture and Microbial Adherence on the Catheters Soaked in Urine Culture Using Test 5 Described Before (7 Days in Urine)

Test organism: C. albicans (10⁵ cfu/ml).

TABLE 17
Effect of curcumin C-3 complex and lubricating
agents on CHX-AgSD coated silicone catheter.

| | Bacterial | Log growth |
|---|---|---|
| | colonies | on 2 cm |
| | (cfu/ml) in | catheter |
| Groups | urine culture | segment |
| Uncoated catheter (Control) | 10⁶-10⁷ | 7.0 |
| 21 | 0 | 0 |
| 22 | 0 | 0 |
| 23 | 0 | 0 |
| 24 | 10²-10³ | 2.0 |

Conclusion: Silicone catheters coated with CHX—CUR-AgSD were more lubricious and showed no C. albicans adherence on the catheter surface after 7 days.

### Example 23: Comparison of Bacterial Adherence of Clinical Strains of Microbes on Latex Foley Catheters Coated with CHX—CUR-AgSD and Commercial Catheters Using BactiGuard™ (Gold, Silver and Palladium Coating)

Latex Foley catheters were coated with the following compositions in accordance with the techniques provided in Example 16 above:

| | |
|---|---|
| Ingredients (%) | Percentage |

Group 10

| | |
|---|---|
| Chlorhexidine base | 1.0 |
| Mandelic acid | 0.75 |
| Silver sulfadiazine | 0.75 |
| EG-93A | 2.0 |
| Tetrahydrofuran (THF) | 95.5(V) |

Group 12

| | |
|---|---|
| Chlorhexidine base | 1.0 |
| Mandelic acid | 0.75 |
| Curcumin C3-complex | 1.0 |
| Silver sulfadiazine | 0.75 |
| EG-93A | 2.0 |
| Tetrahydrofuran (THF) | 94.5(V) |

A. Evaluation of E. coli Adherence on the Surface of Coated Latex Urinary Catheter and Determination of Bacterial Colonies in Urine Culture in Presence of Coated Catheter.

Test 11:

method of determination of bacterial adherence on coated latex catheter surface (Semi-quantitative evaluation): antimicrobial coated latex urinary catheter segments (1 cm) were placed in a sterile culture tube individually and suspended in 2 ml of urine (collected from a healthy subject) inoculated with multi-drug resistant E. coli (10⁵ cfu/ml) isolated from a urine sample of a patient admitted in Bharati Hospital, Pune, India. The tubes were kept in an incubator at 37° C. overnight. Uncoated catheter segments were used as the control. Triplicate samples from each group were taken for the experiment. For comparison, a commercially available infection resistant urinary catheter “Bactiguard™” (coated with gold, silver and palladium alloy) was also tested. Catheter segments were transferred everyday into fresh urine culture inoculated with E. coli (10⁵ cfu/ml) and continued for 20 days. After 2, 7, 12 and 18 day intervals, the segments were removed from the tube and blotted on tissue paper to drain out the residual urine. Then, they were washed twice in sterile normal saline and blotted to dry. Finally, each of them was rolled on tryptone soya agar (TSA) plate and incubated for 24 hours at 37° C. to evaluate the efficacy of the coated catheters in preventing bacterial adherence on the surface. Presence of viable bacteria (E. coli) colonies in urine culture was also determined at 2, 7, 12 and 18 day intervals.

TABLE 18A
Antimicrobial efficacy after day two.

| | E. coli | E. coli |
|---|---|---|
| | colonies | adherence |
| | (cfu/ml) present | (Log₁₀ cfu/cm |
| Experimental groups | in urine culture | catheter surface) |
| Uncoated | 10⁷-10⁸ | 7.0 |
| CHX + AgSD (Group 10) | 0 | 0.0 |
| CHX + AgSD + CUR (Group 12) | 0 | 0.0 |
| Bactiguard ™ | 10⁷-10⁸ | 6.0 |

TABLE 18B
Antimicrobial efficacy after day seven.

| | E. coli | E. coli |
|---|---|---|
| | colonies | adherence |
| | (cfu/ml) present | (Log₁₀ cfu/cm |
| Experimental groups | in urine culture | catheter surface) |
| Uncoated | >10⁸ | 8.0 |
| CHX + AgSD (Group 10) | 10⁶-10⁷ | 1.76 |
| CHX + AgSD + CUR (Group 12) | 10²-10³ | 0.3 |
| Bactiguard ™ | >10⁸ | 8.0 |

TABLE 18C
Antimicrobial efficacy after day twelve.

| | E. coli | E. coli |
|---|---|---|
| | colonies | adherence |
| | (cfu/ml) present | (Log₁₀ cfu/cm |
| Experimental groups | in urine culture | catheter surface) |
| Uncoated | >10⁸ | 8.0 |
| CHX + AgSD (Group 10) | 10⁷-10⁸ | 1.97 |
| CHX + AgSD + CUR (Group 12) | 10³-10⁴ | 0.6 |
| Bactiguard ™ | >10⁸ | 8.0 |

TABLE 18D
Antimicrobial efficacy after day eighteen.

| | E. coli | E. coli |
|---|---|---|
| | colonies | adherence |
| | (cfu/ml) present | (Log₁₀ cfu/cm |
| Experimental groups | in urine culture | catheter surface) |
| Uncoated | >10⁸ | 8.0 |
| CHX + AgSD (Group 10) | >10⁸ | 4.0 |
| CHX + AgSD + CUR (Group 12) | >10⁸ | 2.3 |

Test 12:

method of determination of bacterial adherence on coated catheter surface (Quantitative evaluation): after 20 days, the catheter segments were removed from the tubes containing urine culture inoculated with E. coli and blotted on tissue paper to drain out the residual urine. They were rinsed twice in 10 ml of sterile normal saline and blotted to dry. Each catheter segment was then put into a culture tube containing 4 ml of DNF (drug neutralizing fluid) and subjected to bath sonication for 20 minutes. After dilution, 0.5 ml aliquot from each tube was spread on TSA plate and incubated at 37° C. for 24 hours.

TABLE 19
Adherence of E. coli on the surface of coated latex urinary
catheter after 20 days soaking in urine culture.

| | E. coli adherence |
|---|---|
| Experimental groups | (Log₁₀ cfu/cm catheter surface) |
| Uncoated | 8.87 |
| CHX + AgSD (Group 10) | 5.78 |
| CHX + AgSD + CUR (Group 12) | 3.6 |
| Bactiguard ™ | 8.18 |

Conclusion:

latex urinary catheters coated with CHX+AgSD+CUR were found to be most effective in preventing the adherence of E. coli (clinical isolate) on its surface after 20 days, whereas uncoated catheter as well as commercially available “Bactiguard™” infection resistant urinary catheter showed a heavy bacterial adherence on its surface after 2 days.

B. Evaluation of C. albicans Adherence on the Surface of Coated Latex Urinary Catheter and Determination of C. albicans Colonies in Urine Culture in Presence of Coated Catheter.

Test 13:

method of determination of C. albicans adherence on coated latex catheter surface (Semi-quantitative evaluation): the test method is similar as described in Test 11. Antimicrobial coated latex urinary catheter segments (1 cm) were suspended in 2 ml of urine culture inoculated with a clinical isolate of C. albicans (10⁵ cfu/ml), collected from Bharati Hospital, Pune, India. The tubes were kept in an incubator at 30° C. overnight. Catheter segments were transferred everyday into fresh urine culture inoculated with C. albicans (10⁵ cfu/ml) and continued for 18 days. After 2, 7, 13 and 16 day intervals, the segments were removed and washed twice in sterile normal saline. Finally, each of them was rolled on TSA plate and incubated for 24-48 hours at 30° C. to evaluate the efficacy of the coated catheters in preventing the adherence of C. albicans on the surface. The presence of viable C. albicans colonies in urine culture was also determined at 2, 7, 13 and 16 day intervals.

TABLE 20A
Antimicrobial efficacy after day two.

| | C. albicans | C. albicans |
|---|---|---|
| | colonies | adherence |
| | (cfu/ml) present | (Log₁₀ cfu/cm |
| Experimental groups | in urine culture | catheter surface) |
| Uncoated | 10⁷-10⁸ | 8.0 |
| CHX + AgSD (Group 10) | 0 | 0.0 |
| CHX + AgSD + CUR (Group 12) | 0 | 0.0 |
| Bactiguard ™ | 10⁷-10⁸ | 7.5 |

TABLE 20B
Antimicrobial efficacy after day seven.

| | C. albicans | C. albicans |
|---|---|---|
| | colonies | adherence |
| | (cfu/ml) present | (Log₁₀ cfu/cm |
| Experimental groups | in urine culture | catheter surface) |
| Uncoated | >10⁸ | 8.5 |
| CHX + AgSD (Group 10) | 10³-10⁴ | 0.3 |
| CHX + AgSD + CUR (Group 12) | 10³-10⁴ | 0.0 |
| Bactiguard ™ | >10⁸ | 8.5 |

TABLE 20C
Antimicrobial efficacy after day thirteen.

| | C. albicans | C. albicans |
|---|---|---|
| | colonies | adherence |
| | (cfu/ml) present | (Log₁₀ cfu/cm |
| Experimental groups | in urine culture | catheter surface) |
| Uncoated | >10⁸ | 9.0 |
| CHX + AgSD (Group 10) | >10⁸ | 2.0 |
| CHX + AgSD + CUR (Group 12) | 10⁴-10⁵ | 0.3 |
| Bactiguard ™ | >10⁸ | 9.0 |

TABLE 20D
Antimicrobial efficacy after day 16

| | C. albicans | C. albicans |
|---|---|---|
| | colonies | adherence |
| | (cfu/ml) present | (Log₁₀ cfu/cm |
| Experimental groups | in urine culture | catheter surface) |
| Uncoated | >10⁸ | 9.0 |
| CHX + AgSD (Group 10) | >10⁸ | 4.0 |
| CHX + AgSD + CUR (Group 12) | >10⁸ | 2.85 |

Test 14:

method of determination of C. albicans adherence on coated catheter surface (quantitative evaluation): the test method is similar as described in Test 12. After 18 days, the catheter segments were removed, rinsed twice in sterile normal saline and subjected to bath sonication for 20 minutes in 4 ml of DNF. After dilution, 0.5 ml aliquot from each tube was spread on TSA plate and incubated at 30° C. for 24-48 hours.

TABLE 21
Adherence of C. albicans on the surface of coated latex
urinary catheter after 18 days soaking in urine culture.

| | C. albicans adherence |
|---|---|
| Experimental groups | (Log₁₀ cfu/cm catheter surface) |
| Uncoated | 9.25 |
| CHX + AgSD (Group 10) | 5.63 |
| CHX + AgSD + CUR (Group 12) | 4.5 |
| Bactiguard ™ | 8.87 |

Conclusion:

latex urinary catheters coated with CHX+AgSD+CUR were found to be most effective in preventing the adherence of C. albicans (clinical isolate) on its surface even after 18 days, whereas uncoated catheter as well as commercially available “Bactiguard™” infection resistant urinary catheter showed a heavy bacterial adherence on its surface after 2 days.

C. Evaluation of P. aeruginosa Adherence on the Surface of Coated Latex Urinary Catheter and Determination of P. aeruginosa Colonies in Urine Culture in Presence of Coated Catheter.

Test 15:

method of determination of P. aeruginosa adherence on coated latex catheter surface (semi-quantitative evaluation): the test method is similar as described in Test 11. Antimicrobial coated latex urinary catheter segments (1 cm) were suspended in 2 ml of urine culture inoculated with multi-drug resistant clinical isolate of P. aeruginosa (10⁵ cfu/ml), collected from Bharati Hospital, Pune, India. The tubes were kept in an incubator at 37° C. overnight. Catheter segments were transferred everyday into fresh urine culture inoculated with P. aeruginosa (10⁵ cfu/ml) and continued for 8 days. After 2, 6 and 7 day intervals, the segments were removed and washed twice in sterile normal saline. Each of them was then rolled on a TSA plate and incubated for 24 hours at 37° C. to evaluate the efficacy of coated catheters in preventing the adherence of P. aeruginosa on the surface. Presence of viable P. aeruginosa colonies in urine culture was also determined at 2, 6 and 7 day intervals.

TABLE 22A
Antimicrobial efficacy after day two.

| | P. aeruginosa | P. aeruginosa |
|---|---|---|
| | colonies | adherence |
| | (cfu/ml) present | (Log₁₀ cfu/cm |
| Experimental groups | in urine culture | catheter surface) |
| Uncoated | 10⁶-10⁷ | 7.5 |
| CHX + AgSD (Group 10) | 0 | 0.0 |
| CHX + AgSD + CUR (Group 12) | 0 | 0.0 |
| Bactiguard ™ | 10⁶-10⁷ | 7.5 |

TABLE 22B
Antimicrobial efficacy after day six.

| | P. aeruginosa | P. aeruginosa |
|---|---|---|
| | colonies | adherence |
| | (cfu/ml) present | (Log₁₀ cfu/cm |
| Experimental groups | in urine culture | catheter surface) |
| Uncoated | >10⁸ | 8.0 |
| CHX + AgSD (Group 10) | >10⁸ | 1.6 |
| CHX + AgSD + CUR (Group 12) | >10⁸ | 1.5 |
| Bactiguard ™ | >10⁸ | 8.0 |

TABLE 22C
Antimicrobial efficacy after day seven.

| | P. aeruginosa | P. aeruginosa |
|---|---|---|
| | colonies | adherence |
| | (cfu/ml) present | (Log₁₀ cfu/cm |
| Experimental groups | in urine culture | catheter surface) |
| Uncoated | >10⁸ | 8.0 |
| CHX + AgSD (Group 10) | >10⁸ | 3.0 |
| CHX + AgSD + CUR (Group 12) | >10⁸ | 3.0 |

Test 16:

method of determination of P. aeruginosa adherence on coated catheter surface (Quantitative evaluation): the test method is similar as described in Test 12. After 8 days, the catheter segments were removed, rinsed twice in sterile normal saline and subjected to bath sonication for 20 minutes in 4 ml of DNF. After dilution, a 0.5 ml aliquot from each tube was spread on TSA plate and incubated at 37° C. for 24 hours.

TABLE 23
Adherence of P. aeruginosa on the surface of coated latex
urinary catheter after 8 days soaking in urine culture.

| | P. aeruginosa adherence |
|---|---|
| Experimental groups | (Log₁₀ cfu/cm catheter surface) |
| Uncoated | 8.47 |
| CHX + AgSD (Group 10) | 5.41 |
| CHX + AgSD + CUR (Group 12) | 5.28 |
| Bactiguard ™ | 8.28 |

Conclusion:

latex urinary catheters coated with either CHX+AgSD or CHX+AgSD+CUR was found to be effective in preventing the adherence of P. aeruginosa (clinical isolate) on the surface even after 7 days, whereas uncoated catheters as well as commercially available “Bactiguard™” infection resistant urinary catheters showed a heavy bacterial adherence on its surface after 2 days.

### Example 24: Effect of 1,2-Decanediol as the Lubricating Agent on Adherence of Anti-Microbial Coating Compositions on the Latex Catheter Surface

### A. Method of Preparation of 100 ml CHX—CUR-AgSD Coating Solution Containing 1,2-Decanediol.

One gm of chlorhexidine (CHX), 0.75 gm of mandelic acid and 2.0 gm of 1,2-decanediol were taken in a glass container. Ten ml of methanol was added into it and vortexed well to dissolve the CHX, mandelic acid and 1,2-decanediol. To this mixture, 50 ml of 6% EG-93A polymer, dissolved in tetrahydrofuran (THF), was added and mixed well. Finally, 1.0 gm of curcumin-C3 complex (CUR) was added and mixed well to produce a bright yellow colored solution containing CHX, CUR, 1,2-decanediol and EG-93A polymer.

In a separate container, 0.75 g of silver sulfadiazine (AgSD) was taken and 10 ml of THF was added to make a uniform paste of AgSD and then 10 ml of THF was added to make a uniform suspension, which was transferred to the solution containing the mixture of CHX, CUR, 1,2-decanediol and EG-93A polymer, using a pipette. The residual AgSD was rinsed with 14.5 ml of THF, transferred to the same mixture solution, and mixed well. Thus, CHX—CUR—AgSD-1,2-decanediol coating solution was prepared.

The following groups were prepared. (V) indicates measured by volume.

| | |
|---|---|
| Ingredients (%) | Percentage |

Group 25

| | |
|---|---|
| Chlorhexidine base | 1.0 |
| Mandelic acid | 0.75 |
| Methanol | 10.0 (V) |
| Curcumin C3-complex | 1.0 |
| Silver sulfadiazine | 0.75 |
| EG-93A | 3.0 |
| Tetrahydrofuran (THF) | 83.5 (V) |

Group 26

| | |
|---|---|
| Chlorhexidine base | 1.0 |
| Mandelic acid | 0.75 |
| 1,2-Decanediol | 2.0 |
| Methanol | 10.0 (V) |
| Curcumin C3-complex | 1.0 |
| Silver sulfadiazine | 0.75 |
| EG-93A | 3.0 |
| Tetrahydrofuran (THF) | 81.5 (V) |

Latex urinary catheters were soaked in the above-mentioned coating solutions for 1 minute. After 1 minute, the catheter was removed from the coating solution and allowed to dry, overnight at room temperature, and tested as follows.

Test 17:

stability of coating on the catheter bulb. The adherence of the anti-microbial coating composition on the latex catheter surface was tested by inflating the coated latex catheter bulbs (Group 25 & Group 26) with water.

Conclusion:

the adherence of coating composition on the catheter surface was found to be more durable when 1, 2-decanediol was present in the coating solution.

Test 18:

evaluation of bacterial colonies in urine culture and bacterial adherence on the surface of above coated catheters soaked in urine culture was performed by following Test 15 method described above, using P. aeruginosa (10⁵ cfu/ml) as the test organism. The efficacy of coated catheters in preventing the adherence of P. aeruginosa on its surface was evaluated after 2, 4, 5 and 6 day intervals. Presence of viable P. aeruginosa colonies in urine culture also were determined at 2, 4, 5 and 6 day intervals.

TABLE 24
Antimicrobial efficacy of catheters coated
with and without decanediol after day six.

| | P. aeruginosa | P. aeruginosa |
|---|---|---|
| | colonies | adherence |
| | (cfu/ml) present | (Log₁₀ cfu/cm |
| Experimental groups | in urine culture | catheter surface) |
| Uncoated | >10⁸ | 8.0 |
| CHX + AgSD + CUR (Group 25) | >10⁸ | 2.0 |
| CHX + AgSD + CUR + 1,2- | >10⁸ | 2.2 |
| decanediol (Group 26) | | |

Conclusion:

latex urinary catheters coated with either CHX+AgSD+CUR or CHX+AgSD+CUR+1,2-decanediol was found to show similar efficacy.

### Example 25: Effect of Octoxyglycerin as the Lubricating Agents to Evaluate Bacterial Adherence on the Surface of Coated Latex Catheter

Production of Coating Compositions.

The following coating compositions were prepared according to Example 24 except that 2.0% decanediol was replaced with 1.0% octoxyglycerin (lubricating agent) in the coating solution. Catheters were soaked for 1 minute in the coating solution, removed at a steady speed and dried at room temperature overnight. Group 27: 1.0% CHX+1.0% CUR+0.75% AgSD+3% EG-93A+1.0% octoxyglycerin.

TABLE 25
Antimicrobial efficacy after day six.

| | P. aeruginosa | P. aeruginosa |
|---|---|---|
| | colonies | adherence |
| | (cfu/ml) present | (Log₁₀ cfu/cm |
| Experimental groups | in urine culture | catheter surface) |
| Uncoated | >10⁸ | 8.0 |
| CHX + AgSD + CUR (Group 25) | >10⁸ | 2.1 |
| CHX + AgSD + CUR + | >10⁸ | 2.3 |
| Octoxyglycerin (Group 27) | | |
| CHX + AgSD + CUR + 1,2- | >10⁸ | 2.2 |
| decanediol (Group 26) | | |

Conclusion:

Addition of lubricants did not affect the antimicrobial efficacy in the groups containing curcumin.

### Example 26. Preparation of Coating Solution Containing CHX-AgSD-CUR-1,2

decanediol (chlorhexidine, silver sulfadiazine, curcumin C3-complex and 1,2-decanediol) for silicone Foley catheters. The method of preparing 100 ml CHX—CUR-AgSD coating solution containing 1,2-Decanediol was as follows. One gm of chlorhexidine (CHX), 0.75 gm of mandelic acid and 2.0 gm of 1,2-decanediol were placed in a glass container. Ten ml of methanol was added to it and vortexed well to dissolve the CHX, mandelic acid and 1,2-decanediol. To this mixture, 24 ml of 8.3% polymer mixture [EG 93A+EG 60D (4:1) dissolved in tetrahydrofuran (THF)] was added and mixed well by vortex. One gm of curcumin-C3 complex (CUR) was added into this and mixed well to produce a bright yellow colored CHX—CUR-1,2-decanediol mixture in a polymer solution. To this solution, 3.0 gm of silicone medical adhesive Type A-100 or Silicone adhesive MD7-4502 was added and mixed well.

For coating silicone urinary Foley catheters, the above CHX—CUR-AgSD-1,2 decanediol coating solution was transferred into a glass cylinder. The silicone urinary catheter was soaked in the coating solution for 30 seconds, continuously swirling the catheter in a swift motion to prevent AgSD from settling down. After 30 seconds, the catheter was removed at a constant steady speed from the coating solution and allowed to dry for overnight at 37° C. in an incubator.

The following groups were prepared.

Group 28

| Ingredients (%) | Percentage |
|---|---|
| Chlorhexidine base | 1.0 |
| Mandelic acid | 0.75 |
| 1,2-Decanediol | 2.0 |
| Methanol | 10.0 (V) |
| EG-93A | 1.6 |
| EG-60D | 0.4 |
| Medical adhesive Type A-100/MD7-4502 | 3.0 |
| Silver sulfadiazine | 0.75 |
| Tetrahydrofuran (THF) | 80.5 (V) |

Group 29

| Ingredients (%) | Percentage (%) |
|---|---|
| Chlorhexidine base | 1.0 |
| Mandelic acid | 0.75 |
| 1,2-Decanediol | 2.0 |
| Methanol | 10.0 (V) |
| Curcumin C3-complex | 1.0 |
| EG-93A | 1.6 |
| EG-60D | 0.4 |
| Medical adhesive Type A-100/MD7-4502 | 3.0 |
| Silver sulfadiazine | 0.75 |
| Tetrahydrofuran (THF) | 79.5 (V) |

(V) indicates measured by volume.

### Example 27: Evaluation of P. aeruginosa Adherence on the Surface of Coated Silicone Urinary Catheter and Determination of P. aeruginosa Colonies in Urine Culture in Presence of Coated Catheters

Test 19:

evaluation of bacterial colonies in urine culture and bacterial adherence on the surface of coated silicone catheters soaked in urine culture inoculated with P. aeruginosa (Semi-quantitative evaluation), using P. aeruginosa (10⁵ cfu/ml) as the test organism. The test method is similar as described in Test 15. The efficacy of coated silicone catheter in preventing the adherence of P. aeruginosa on its surface was evaluated after 2, 6, 11 and 15 day intervals. Presence of viable P. aeruginosa colonies in urine culture also was determined at 2, 6, 11 and 15 day intervals.

TABLE 26A
Antimicrobial efficacy after day two.

| | P. aeruginosa | P. aeruginosa |
|---|---|---|
| | colonies | adherence |
| | (cfu/ml) present | (Log₁₀ cfu/cm |
| Experimental groups | in urine culture | catheter surface) |
| Uncoated | 10⁶-10⁷ | 7.5 |
| CHX + AgSD (Group 28) | 0 | 0.0 |
| CHX + AgSD + CUR (Group 29) | 0 | 0.0 |

TABLE 26B
Antimicrobial efficacy after day six.

| | P. aeruginosa | P. aeruginosa |
|---|---|---|
| | colonies | adherence |
| | (cfu/ml) present | (Log₁₀ cfu/cm |
| Experimental groups | in urine culture | catheter surface) |
| Uncoated | >10⁸ | 8.0 |
| CHX + AgSD (Group 28) | >10⁷ | 0.0 |
| CHX + AgSD + CUR (Group 29) | 10²-10³ | 0.0 |

TABLE 26C
Antimicrobial efficacy after day eleven.

| | P. aeruginosa | P. aeruginosa |
|---|---|---|
| | colonies | adherence |
| | (cfu/ml) present | (Log₁₀ cfu/cm |
| Experimental groups | in urine culture | catheter surface) |
| Uncoated | >10⁸ | 8.0 |
| CHX + AgSD (Group 28) | >10⁸ | 0.0 |
| CHX + AgSD + CUR (Group 29) | >10⁸ | 0.0 |

TABLE 26D
Antimicrobial efficacy after day fifteen.

| | P. aeruginosa | P. aeruginosa |
|---|---|---|
| | colonies | adherence |
| | (cfu/ml) present | (Log₁₀ cfu/cm |
| Experimental groups | in urine culture | catheter surface) |
| Uncoated | >10⁸ | 8.0 |
| CHX + AgSD (Group 28) | >10⁸ | 3.25 |
| CHX + AgSD + CUR (Group 29) | >10⁸ | 2.5 |

Test 20:

method of determination of P. aeruginosa adherence on coated silicone catheter surface (Quantitative evaluation): the test method is similar to Test 12. After 16 days, the catheter segments were removed, rinsed twice in sterile normal saline and subjected to bath sonication for 20 minutes in 4 ml of DNF. After dilution, a 0.5 ml aliquot from each tube was spread on a TSA plate and incubated at 37° C. for 24 hours.

TABLE 27
Adherence of P. aeruginosa on the surface of coated silicone
urinary catheter after 16 days soaking in urine culture.

| | P. aeruginosa adherence |
|---|---|
| Experimental groups | (Log₁₀ cfu/cm catheter surface) |
| Uncoated | 8.76 |
| CHX + AgSD (Group 28) | 5.62 |
| CHX + AgSD + CUR (Group 29) | 4.63 |

Conclusion:

silicone urinary catheters coated with CHX+AgSD+CUR were found to be most effective in preventing the adherence of P. aeruginosa (clinical isolate) on the surface even after 16 days, whereas uncoated catheters showed a heavy bacterial adherence on the surface after 2 days.

### REFERENCES

All references listed below and throughout the specification are hereby incorporated by reference in their entirety.
1. Drekonja et al., Antimicrobial Urinary Catheters: a systematic review. Expert Rev. Med. Devices 5(4):495-506, 2008.
2. Islas et al., Singly and Binary Grafted Poly(vinyl chloride) Urinary Catheters that Elute Ciprofloxacin and Prevent Bacteria Adhesion. Int. J. Pharm. 488(1-2):20-28, 2015.
3. U.S. Pat. No. 8,932,624.
4. United States Patent Publication No. 2012-0201902.
5. United States Patent Publication No. 2011-002929.
6. International Patent Publication No. WO/2000/015288A1.
7. International Patent Publication No. WO/2012/109187A1.
8. U.S. Pat. No. 7,829,029.
9. International Patent Publication No. WO/2008/031601A1.
10. International Patent Publication No. WO/2002/051464A2.
11. Gaonkar et al., Efficacy of a Silicone Urinary Catheter Impregnated with Chlorhexidine and Triclosan against Colonization with Proteus mirabilis and Other Uropathogens. 28(5):596-598, 2007
12. Gaonkar et al., Evaluation of the Antimicrobial Efficacy of Urinary Catheters Impregnated with Antiseptics in an In Vitro Urinary Tract Model. 24(7):506-513, 2003.
13. Mody et al., Enhancing Resident Safety by Preventing Healthcare-associated Infection: a national initiative to reduce catheter-associated urinary tract infections in nursing homes. Clin. Infect. Dis. 61(1):86-89, 2015.
14. Thallinger et al., Cellobiose Dehydrogenase Functionalized Urinary Catheter as Novel Antibiofilm System. J. Biomed. Mater. Res. B. Appl. Biomater. Aug. 6, 2015.
15. Adesina et al., Prevention of Bacterial Biofilms Formation on Urinary Catheter by Selected Plant Extracts. Pak. J. Biol. Sci. 18(2):67-73, 2015.
16. Thomas et al., Inhibitory Effect of Silver Nanoparticle Fabricated Urinary Catheter on Colonization Efficiency of Coagulase Negative Staphylococci. J. Photochem. Photobiol. B 149:68-77, 2015.
17. Sladjana et al., Biocide Activity against Urinary Catheter Pathogens. Antimicrob. Agents Chemother. 58(2):1192-1194, 2014.

1. A composition for providing an antibacterial coating, which comprises:
(a) a solvent;
(b) a chlorhexidine compound selected from chlorhexidine base and chlorhexidine salt;
(c) a silver compound selected from elemental silver and silver salt;
(d) a curcumin compound, and an optional lubricity enhancing agent
(e) a pH adjusting compound selected from an acid; and
(f) a biomedial polymer.

2. The composition of claim 1, wherein the solvent is tetrahydrofuran (THF) or THF and an alkanol.

3. The composition of claim 2, wherein the alkanol is selected from methanol, ethanol, propanol, isopropanol, 1,3-propanediol, 2-methyl-2 propanol, hexanol, or any combination thereof.

4. The composition of claim 2, wherein the solvent is THF.

5. The composition of claim 2, wherein the solvent is a combination of THF and methanol.

6. The composition of claim 1, wherein the chlorhexidine compound is chlorhexidine base.

7. The composition of claim 1, wherein the silver compound is a silver salt.

8. The composition of claim 7, wherein the silver salt is silver sulfadiazine.

9. The composition of claim 1, wherein the curcumin compound is curcumin.

10. The composition of claim 1, wherein the curcumin compound is curcumin-C3 complex.

11. The composition of claim 1, wherein the optional lubricity enhancing agent is selected from a natural oil, silicone oil, an emollient solvent, a hydrogel, and any combination thereof.

12. The composition of claim 11, wherein the natural oil is selected from flax seed oil, grape seed oil, cranberry oil, avocado oil and any combination thereof.

13. The composition of claim 11, wherein the emollient solvent is selected from octoxyglycerin, an alkanediol, and any combination thereof.

14. The composition of claim 13, wherein the alkanediol is 1,2-decanediol.

15. The composition of claim 11, wherein the hydrogel is selected from polyethylene oxide and polyethylene glycol, or any combination thereof.

16. The composition of claim 1 wherein the acid is selected from mandelic acid and lactic acid and any combination thereof.

17. The composition of claim 1, wherein the biomedical polymer is selected from polyurethane EG-93A, polyurethane EG-60D, silicone adhesive Type A, silicone adhesive MD7-4502 and any combination thereof.

18. A composition for providing antimicrobial coating, which contains 60-85% v/v tetrahydrofuran, 10-30% v/v alkanol, 0.5-3.0% w/v chlorhexidine base, 0.2-1.0% w/v silver salt, 1.0-7.0 v/v % natural oil and 1.0-5.0 w/v % biomedical polymer.

19. The composition of claim 1, which contains a chlorhexidine compound, a curcumin compound, silver sulfadiazine, mandelic acid and optional lubricating agent 1,2-decanediol.

20. The composition of claim 1, which contains 60-90% v/v tetrahydrofuran, 0-30% v/v alkanol, 0.5-3.0% w/v chlorhexidine base, 0.25-2.0% w/v curcumin, 0.2-1.0% w/v silver salt, and 1-5% w/v biomedical polymer, an optional lubricating agent 0.5-3.0% w/v 1,2-decanediol.

21. The composition of claim 1, in which the ratio of chlorhexidine compound to curcumin compound is 1:1 to 1:2.

22. An ointment, lotion, or wound healing cream comprising the composition of claim 1.

23. An article coated with the composition of claim 1.

24. The article of claim 23, which is a medical device.

25. The article of claim 24, which is a catheter.

26. The article of claim 25, which is a urinary catheter.

27. The article of 25 which is a central venous catheter.

28. The article of 25 which is an endotracheal tube.

29. The article of claim 24, which is made of latex, silicone, polyurethane or polyvinyl chloride.

30. A method of making an article of claim 23, which comprises soaking the article in a composition of claim 1.

31. The method of claim 26, which comprises soaking the article in a composition of claim 1 for 30 seconds to 2 minutes.

32. A method of making the composition of claim 1, which comprises the steps of:
(a) mixing together chlorhexidine base, mandelic acid and optional lubricating agent 1,2 decanediol
(b) adding methanol to the mixture of (a) to dissolve the ingredients
(c) adding a biomedical polymer EG 93A, dissolved in THF to the mixture of (b);
(d) adding curcumin C3 complex to the mixture of (c)
(e) in a separate container, mixing a silver salt and THF to form a silver mixture;
(f) adding the silver mixture of (e) to the mixture of (d).

33. A method of making the composition providing antimicrobial coating, which comprises the steps of:
(a) mixing chlorhexidine base in alkanol;
(b) adding biomedical polymer dissolved in tetrahydrofuran to the mixture of (a);
(c) in a separate container, mixing silver nitrate and tetrahydrofuran to form a silver mixture, and adding ammonium hydroxide to a final concentration of 0.5% to form a clear solution;
(d) adding the silver mixture of (c) to the mixture of (b); and
(e) optionally adding a lubricating agent to the mixture of step (a).

34. A method of making the composition of claim 18, which comprises the steps of:
(a) mixing chlorhexidine base in methanol in a container;
(b) adding a biomedical polymer dissolved in tetrahydrofuran to the mixture of (a);
(c) adding a natural oil to the mixture of (b);
(d) in a separate container, mixing a silver salt and tetrahydrofuran to form a silver mixture; and
(e) adding the silver mixture of (d) to the mixture of (c).

35. A method of making the composition of claim 1, comprising the steps of:
(a) mixing chlorhexidine base and curcumin-C3 complex in a container;
(b) adding a biomedical polymer mixture of EG 93A & EG 60D, dissolved in tetrahydrofuran, to the mixture of (a);
(c) adding mandelic acid or lactic acid to the mixture of (b) to adjust the pH to 6.0-7.0;
(d) adding silicone medical adhesive Type A or MD7-4502 to the mixture of (c);
(e) in a separate container, mixing a silver salt and tetrahydrofuran to form a silver mixture; and
(f) adding the silver mixture of (e) to the mixture of (d).

36. A method of making the composition of claim 1, which comprises the steps of:
(a) mixing together chlorhexidine base, mandelic acid optional lubricating agent 1,2 decanediol;
(b) adding methanol to the mixture of (a) to dissolve the ingredients
(c) adding a biomedical polymer mixture of EG 93A & EG 60D, dissolved in THF to the mixture of (b);
(d) adding curcumin C3 complex to the mixture of (c)
(e) adding silicone medical adhesive Type A or MD7-4502 to the mixture of (d)
(f) in a separate container, mixing a silver salt and THF to form a silver mixture;
(g) adding the silver mixture of (f) to the mixture of (e).

37. A method of making the composition of claim 1, which comprises the steps of:
(a) mixing together chlorhexidine base and a curcumin compound;
(b) adding to the mixture of (a) a biomedical polymer EG 93A dissolved in THF;
(c) adding mandelic acid or lactic acid to the mixture of (b) to adjust the pH to 6.5-7.0;
(d) in a separate container, mixing a silver salt and THF to form a silver mixture; and
(e) adding the silver mixture of (d) to the mixture of (c).
